(19) United States
(12) Patent Application Publication
(10) Pub. No.: US 2026/0152450 A1
(43) Pub. Date: Jun. 4, 2026

(54) USE OF A PRIMER AND A BIOFERTILIZER TO ENHANCE NITROGEN FIXATION AND/OR ROOT COLONIZATION

(71) Applicant: KULA BIO, INC., Natick, MA (US)

(72) Inventors: Darian SMERCINA, Towson, MD (US); Frederic KENDIRGI, Fort Collins, CO (US); Kyle CARTER, Natick, MA (US); Mélanie IKEH, Framingham, MA (US); Matthew DOMMEL, Waltham, MA (US)

(21) Appl. No.: 19/406,473

(22) Filed: Dec. 2, 2025

Related U.S. Application Data
(60) Provisional application No. 63/726,901, filed on Dec. 2, 2024.

(51) Int. Cl. C05F 17/20; C05C 11/00; C05D 1/02; C05D 9/02; C05F 5/00; C12N 1/205; C12R 1/01

(52) U.S. Cl. CPC C05F 17/20; C05C 11/00; C05D 1/02; C05D 9/02; C05F 5/00; C12N 1/205; C12R 2001/01

(57) ABSTRACT

The disclosure provides compositions and kits comprising i) a biofertilizer comprising a microorganism, wherein the microorganism is Xanthobacter autotrophicus, and ii) a primer of nitrogen fixation, wherein the primer is optionally glucose, fructose, or a combination thereof. In some aspects, disclosed herein are methods of using a biofertilizer and a primer of nitrogen fixation for priming nitrogen fixation, enhancing exopolysaccharides (EPS) production of a biofertilizer, enhancing microbial root colonization of a plant, and/or increasing viability of a biofertilizer.

### CROSS REFERENCE TO RELATED APPLICATIONS

The present application claims the priority benefit of U.S. Provisional Application No. 63/726,901, filed Dec. 2, 2024, which is hereby incorporated by reference in its entirety.

### FIELD OF THE DISCLOSURE

The disclosure relates to nitrogen-fixing microbial biofertilizers and compounds that act as a primer for the nitrogen-fixing microbe's activity.

### BACKGROUND OF THE DISCLOSURE

Agricultural systems depend on the global nitrogen cycle and the application of human-produced nitrogen for sustained productivity. In fact, over 80% of all human-produced nitrogen is used for agriculture in an effort to maximize yields and meet the demands of an increasing global population (Smil, V., Nature 400:415 (1999); Tilman, D., et al., PNAS 108:20260-20264 (2002); Campbell, B. M., et al., Ecology and Society 22(4):8 (2017)).

However, of the approximately 160 Tg of nitrogen applied to agricultural systems annually (Galloway, J. N., et al., Science 320:889-892 (2008)), only around 40% is effectively utilized by crops (Anas, M., et al., Biological Research 53:1-20 (2020)). The remaining 60% is lost through various pathways, with significant negative environmental impacts. Excess nitrogen is denitrified to the atmosphere, contributing nearly 50% of all global N₂O emissions (Shcherbak, I., et al., PNAS 111:9199-9204 (2014)). Excess nitrogen can also run off or leach into nearby waterways, causing eutrophication, harming aquatic life, and reducing water quality (Tilman, D., et al., Nature 418:671-677 (2002)).

Nitrogen-fixing microbial biofertilizers are a sustainable alternative to synthetic fertilizers. Microbial biofertilizers harness the capacity of bacteria to convert gaseous nitrogen (N₂) into biologically available ammonia (NH₃) through the process of nitrogen fixation. Microbial fertilizers may also be more efficient than synthetic fertilizers. Croplands relying on nitrogen derived from microbial nitrogen fixation tend to be more efficient than those relying heavily on synthetic nitrogen inputs (Lassaletta, L., et al., Environmental Research Letters 9:105011 (2014); Udvardi, M., et al., Frontiers in Sustainable Food Systems 5:660155 (2021)).

Accordingly, a need exists for improved nitrogen-fixing microbial biofertilizers. Such biofertilizers should function in planta and demonstrate the ability to associate with diverse plant species.

### BRIEF SUMMARY

Certain aspects of the disclosure provide a composition comprising i) a biofertilizer comprising a microorganism, wherein the microorganism is Xanthobacter autotrophicus, and ii) a primer of nitrogen fixation, wherein the primer is glucose, fructose, sucrose, malic acid, pantothenic acid, thiamine, an amino acid, potassium chloride, sodium molybdate, molasses, or a combination thereof.

Certain aspects of the disclosure provide a composition comprising i) a biofertilizer comprising a microorganism, wherein the microorganism is Xanthobacter autotrophicus, and ii) a primer of nitrogen fixation.

Certain aspects of the disclosure provide a composition comprising i) a biofertilizer comprising a microorganism, and ii) a primer of nitrogen fixation, wherein the primer is fructose, glucose, sucrose, malic acid, pantothenic acid, thiamine, an amino acid, potassium chloride, sodium molybdate, molasses, or a combination thereof.

In some aspects, the primer comprises a carbohydrate.

In some aspects, the carbohydrate is a sugar. In some aspects, the primer comprises a source of the carbohydrate (e.g., molasses).

In some aspects, the sugar is sucrose, mannose, arabinose, glucose, fructose, or combinations thereof.

In some aspects, the primer is glucose.

In some aspects, the primer is fructose.

In some aspects, the sugar is sucrose.

In some aspects, the carbohydrate is molasses.

In some aspects, the primer comprises an amino acid.

In some aspects, the amino acid is glycine.

In some aspects, the amino acid is glycine, glutamic acid, arginine, asparagine, tryptophan, or any combination thereof.

In some aspects, the amino acid is glutamic acid, glycine, or tryptophan.

In some aspects, the amino acid is glutamic acid, histidine, glycine, arginine, or any combination thereof.

In some aspects, the primer comprises one or more iron-rich soluble compounds.

In some aspects, the primer comprises one or more molybdenum rich-soluble compounds.

In some aspects, the molybdenum rich-soluble compound is sodium molybdate.

In some aspects, the primer comprises an organic acid.

In some aspects, the organic acid is malic acid.

In some aspects, the primer is a salt solution.

In some aspects, the salt solution is potassium chloride.

In some aspects, the primer is pantothenic acid, thiamine, or a combination thereof.

In some aspects, the primer comprises a source of glycine.

In some aspects, the primer comprises a source of one or more amino acids.

In some aspects, the primer comprises a source of one or more iron-rich soluble compounds.

In some aspects, the primer comprises a source of one or more molybdenum rich-soluble compounds.

In some aspects, the primer is glucose.

In some aspects, the primer is fructose.

In some aspects, the microorganism comprises or is selected from the group consisting of Acidiphilium species, Alcaligenes species, Arthrobacter species, Azohydromonas species, Azospirillum species, Azotobacter species, Bacillus species, Beggiatoa species, Beijerinckia species, Bradyrhizobium species, Burkholderia species, Cupriavidus species, Derxia species, Herbaspirillum species, Hydrogenophaga species, Lactobacillus species, Mesorhizobium species, Methylibium species, Methylocapsa species, Methyloferula species, Methyloversatilis species, Microcyclus species, Nitrosococcus species, Nocardia species, Oligotropha species, Pannonibacter species, Paracoccus species, Pelagibaca species, Pseudomonas species, Pseudooceanicola species, Ralstonia species, Renobacter species, Rhizobium species, Rhodobacter species, Rhodomicrobium species, Rubrivivax species, Salipiger species, Sinorhizobium species, Skermanella species, Stappia species, Thauera species, Variovorax species, Xanthobacter species, and combinations thereof.

In some aspects, the microorganism comprises or is selected from the group consisting of Acidiphilium multivorum, Alcaligenes paradoxus, Azoarcus indigens, Azohydromonas australica, Azohydromonas lata, Azorhizobium caulinodans, Azospirillium brasiliense, Azospirillum amazonsense, Azospirillum lipoferum, Azospirillum lipoferum (RSAL0111), Azospirillum thiophilum, Azotobacter chroococum (MCC 0055), Azotobacter vinelandii, Azotobacter vinelandii (RSAV006), Bacillus megaterium, Bacillus pumilus, Bacillus amyloliquefaciens, Bacillus licheniformis, Bacillus subtilis, Beggiatoa alba, Beijerinckia mobilis, Bradyrhizobium elnakii, Bradyrhizobium japonicum, Bradyrhizobium japonicum (strain USDA 122), Burkholderia vietnameiensis, Cupriavidus necator, Derxia gummosa, Gluconacetobacter diazotrophicus, Gluconacetobacter diazotrophicus (MCC 0046), Herbaspirillum autrotrophicum, Herbaspirillum frisingense (MCC 0052), Hydrogenophaga pseudoflava, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Methylibium petroleiphilum, Methylocapsa aurea, Methyloferula stellate, Methyloversatilis universalis, Microcyclus aquaticus, Microcyclus ebruneus, Nitrosococcus oceani, Nitrosomonas communis, Nitrospirillum amazonense, Nocardia autotrophica, Nocardia opaca, Oligotropha carboxidovorans, Paenibacillus durus (MCC 0046), Pannonibacter phragmitetus, Paracoccus denitrificans, Paracoccus pantrophus, Paracoccus yeei, Pelagibaca bermudensis, Pseudomonas facilis, Pseudomonas fluorescens, Pseudooceanicola atlanticus, Ralstonia eutropha, Renobacter vacuolatum, Rhizobium gallicum, Rhizobium japonicum, Rhizobium japonicum (MCC 0071), Rhizobium leguminosarum, Rhizobium leguminosarum biovar viciae, Rhodobacter capsulatus, Rhodobacter sphaeroides, Rhodomicrobium vannielii, Rubrivivax gelatinosus, Salipiger mucosus, Sinorhizobium americanum, Sinorhizobium fredii, Sinorhizobium meliloti, Skermanella stibiiresistens, Stappia aggregate, Thauera humireducens, Variovorax paradoxus, Xanthobacter autotrophicus, and combinations thereof.

In some aspects, the biofertilizer is in liquid formulation.

In some aspects, the biofertilizer is in a dry formulation.

In some aspects, the primer is in liquid formulation.

In some aspects, the primer is in a dry formulation. In some aspects, the primer is a powder.

In some aspects, the composition comprises about 50 μg to about 150 μg of primer per 1 mL of the biofertilizer.

In some aspects, composition comprises about 0.05 g to about 0.15 g of primer per 1 L, or about 0.05 g to about 10 g of primer per 1 L of the biofertilizer.

In some aspects, the composition comprises about 5% w/v of the primer.

In some aspects, the composition comprises about 2.5% w/v of the primer.

In some aspects, the composition comprises about 21.5 μg/mL of the primer.

In some aspects, the amino acid is arginine.

In some aspects, the composition comprises about 2% w/v to about 3% w/v of the primer, optionally about 2.8% w/v of the primer.

In some aspects, the amino acid is asparagine.

In some aspects, the composition comprises about 0.5% w/v to about 1% w/v of the primer, optionally about 0.6% w/v of the primer.

In some aspects, the amino acid is glutamic acid.

In some aspects, the composition comprises about 10% w/v to about 20% w/v of the primer, optionally about 14.6% w/v of the primer.

In some aspects, the amino acid is glycine.

In some aspects, the composition comprises about 1.5% w/v to about 3% w/v of the primer, optionally about 2.3% w/v of the primer.

In some aspects, the amino acid is tryptophan.

In some aspects, the composition comprises about 0.5% w/v to about 1% w/v of the primer, optionally about 0.7% w/v of the primer.

In some aspects, the amino acid is a combination of the amino acids.

In some aspects, the composition comprises about 15% w/v to about 25% w/v of the primer, optionally about 21% w/v of the primer.

In some aspects, the composition comprises about 25 mg/mL to about 75 mg/mL of the primer, optionally about 50 mg/mL of the primer.

In some aspects, the composition comprises about 0.025 M to about 0.05 M of the primer, optionally about 0.03 M of the primer.

In some aspects, the composition comprises about 0.5 M to about 1.5 M of the primer, optionally about 1.0 M of the primer.

In some aspects, the primer is thiamine.

In some aspects, the composition comprises about 3 μg/mL to about 4 μg/mL of the primer, optionally about 3.3 μg/mL of the primer.

In some aspects, the composition comprises about 6 μg/mL to about 8 μg/mL of the primer, optionally about 6.7 μg/mL of the primer.

In some aspects, the primer is pantothenic acid.

In some aspects, the composition comprises about 2 μg/mL to about 3 μg/mL of the primer, optionally about 2.2 μg/mL of the primer.

In some aspects, the composition comprises about 3 μg/mL to about 5 μg/mL of the primer, optionally about 4.3 μg/mL of the primer.

In some aspects, the composition comprises about 20 μg/mL to about 25 μg/mL of the primer, optionally about 21.5 μg/mL of the primer.

In some aspects, the primer is a combination of thiamine and pantothenic acid.

In some aspects, the composition comprises about 0.7 μg/mL of thiamine and about 4.3 μg/mL of pantothenic acid.

In some aspects, the composition comprises about 0.010 mM to about 0.1 mM of the primer, optionally about 0.016 mM, about 0.064 mM, or about 0.080 mM of the primer.

In some aspects, the composition comprises about 1.0 g/L or about 4.0 g/L of the primer.

Certain aspects of the disclosure provide a kit comprising any of the compositions disclosed herein.

Certain aspects of the disclosure provide a kit comprising i) a biofertilizer comprising a microorganism and ii) a primer of nitrogen fixation.

In some aspects, the primer comprises a carbohydrate.

In some aspects, the carbohydrate is a sugar.

In some aspects, the primer comprises a source of one or more carbohydrates (e.g., molasses).

In some aspects, the sugar is sucrose, mannose, arabinose, glucose, fructose, or any combination thereof.

In some aspects, the primer is glucose.

In some aspects, the primer is fructose.

In some aspects, the sugar is sucrose.

In some aspects, the carbohydrate is molasses.

In some aspects, the primer comprises one or more amino acids.

In some aspects, the primer comprises glycine.

In some aspects, the amino acid is glycine, glutamic acid, arginine, asparagine, tryptophan, or any combination thereof.

In some aspects, the amino acid is glutamic acid, glycine, or tryptophan.

In some aspects, the amino acid is glutamic acid, histidine, glycine, arginine, or any combination thereof.

In some aspects, the amino acid is glutamic acid.

In some aspects, the primer comprises an iron-rich soluble compound.

In some aspects, the primer comprises a molybdenum rich-soluble compound.

In some aspects, the molybdenum rich-soluble compound is sodium molybdate.

In some aspects, the primer comprises an organic acid.

In some aspects, the organic acid is malic acid.

In some aspects, the primer is a salt solution.

In some aspects, the salt solution is potassium chloride.

In some aspects, the primer is pantothenic acid, thiamine, or a combination thereof.

In some aspects, the primer comprises a source of glycine.

In some aspects, the primer comprises a source of one or more amino acids.

In some aspects, the primer comprises a source of one or more iron-rich soluble compounds.

In some aspects, the primer comprises a source of one or more molybdenum rich-soluble compounds.

In some aspects, the microorganism is Xanthobacter autotrophicus.

In some aspects, the microorganism comprises or is selected from the group consisting of Acidiphilium species, Alcaligenes species, Arthrobacter species, Azohydromonas species, Azospirillum species, Azotobacter species, Bacillus species, Beggiatoa species, Beijerinckia species, Bradyrhizobium species, Burkholderia species, Cupriavidus species, Derxia species, Herbaspirillum species, Hydrogenophaga species, Lactobacillus species, Mesorhizobium species, Methylibium species, Methylocapsa species, Methyloferula species, Methyloversatilis species, Microcyclus species, Nitrosococcus species, Nocardia species, Oligotropha species, Pannonibacter species, Paracoccus species, Pelagibaca species, Pseudomonas species, Pseudooceanicola species, Ralstonia species, Renobacter species, Rhizobium species, Rhodobacter species, Rhodomicrobium species, Rubrivivax species, Salipiger species, Sinorhizobium species, Skermanella species, Stappia species, Thauera species, Variovorax species, Xanthobacter species, and combinations thereof.

In some aspects, the microorganism comprises or is selected from the group consisting of Acidiphilium multivorum, Alcaligenes paradoxus, Azoarcus indigens, Azohydromonas australica, Azohydromonas lata, Azorhizobium caulinodans, Azospirillium brasiliense, Azospirillum amazonsense, Azospirillum lipoferum, Azospirillum lipoferum (RSAL0111), Azospirillum thiophilum, Azotobacter chroococum (MCC 0055), Azotobacter vinelandii, Azotobacter vinelandii (RSAV006), Bacillus megaterium, Bacillus pumilus, Bacillus amyloliquefaciens, Bacillus lichenformis, Bacillus subtilis, Beggiatoa alba, Beijerinckia mobilis, Bradyrhizobium elnakii, Bradyrhizobium japonicum, Bradyrhizobium japonicum (strain USDA 122), Burkholderia vietnameiensis, Cupriavidus necator, Derxia gummosa, Gluconacetobacter diazotrophicus, Gluconacetobacter diazotrophicus (MCC 0046), Herbaspirillum autrotrophicum, Herbaspirillum frisingense (MCC 0052), Hydrogenophaga pseudoflava, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Methylibium petroleiphilum, Methylocapsa aurea, Methyloferula stellate, Methyloversatilis universalis, Microcyclus aquaticus, Microcyclus ebruneus, Nitrosococcus oceani, Nitrosomonas communis, Nitrospirillum amazonense, Nocardia autotrophica, Nocardia opaca, Oligotropha carboxidovorans, Paenibacillus durus (MCC 0046), Pannonibacter phragmitetus, Paracoccus denitrificans, Paracoccus pantrophus, Paracoccus yeei, Pelagibaca bermudensis, Pseudomonas facilis, Pseudomonas fluorescens, Pseudooceanicola atlanticus, Ralstonia eutropha, Renobacter vacuolatum, Rhizobium gallicum, Rhizobium japonicum, Rhizobium japonicum (MCC 0071), Rhizobium leguminosarum, Rhizobium leguminosarum biovar viciae, Rhodobacter capsulatus, Rhodobacter sphaeroides, Rhodomicrobium vannielii, Rubrivivax gelatinosus, Salipiger mucosus, Sinorhizobium americanum, Sinorhizobium fredii, Sinorhizobium meliloti, Skermanella stibiiresistens, Stappia aggregate, Thauera humireducens, Variovorax paradoxus, Xanthobacter autotrophicus, and combinations thereof.

In some aspects, the biofertilizer is in liquid formulation.

In some aspects, the biofertilizer is in a dry formulation.

In some aspects, the primer is in liquid formulation.

In some aspects, the primer is in a dry formulation. In some aspects, the primer is a powder.

In some aspects, the kit comprises about 50 μg to about 150 μg of primer per 1 mL of the biofertilizer.

In some aspects, the kit comprises about 0.05 g to about 0.15 g of primer per 1 L, or about 0.05 g to about 10 g of primer per 1 L of the biofertilizer.

Certain aspects of the disclosure provide a method of priming nitrogen fixation, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof, and wherein the microorganism is Xanthobacter autotrophicus.

Certain aspects of the disclosure provide a method of enhancing exopolysaccharides (EPS) production of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof, and wherein the microorganism is Xanthobacter autotrophicus.

Certain aspects of the disclosure provide a method of enhancing microbial root colonization of a plant, comprising i) applying a primer to a biofertilizer comprising a microorganism, and ii) applying the primer and the biofertilizer to a plant, wherein the primer comprises glucose, fructose, sucrose, malic acid, pantothenic acid, thiamine, an amino acid, potassium chloride, sodium molybdate, molasses, or a combination thereof, and wherein the microorganism is Xanthobacter autotrophicus.

Certain aspects of the disclosure provide a method of increasing viability of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof, and wherein the microorganism is Xanthobacter autotrophicus.

In some aspects, the primer comprises glucose.

In some aspects, the primer comprises fructose.

Certain aspects of the disclosure provide a method of priming nitrogen fixation, comprising applying a primer to a biofertilizer comprising a microorganism, wherein the microorganism is Xanthobacter autotrophicus.

Certain aspects of the disclosure provide a method of enhancing exopolysaccharides (EPS) production of a biofertilizer, comprising applying a primer to a biofertilizer comprising a microorganism, wherein the microorganism is Xanthobacter autotrophicus.

Certain aspects of the disclosure provide a method of enhancing microbial root colonization, comprising i) applying a primer to a biofertilizer comprising a microorganism, and ii) applying the primer and the biofertilizer to a plant, wherein the microorganism is Xanthobacter autotrophicus.

Certain aspects of the disclosure provide a method of increasing viability of a biofertilizer, comprising applying a primer to a biofertilizer comprising a microorganism, wherein the microorganism is Xanthobacter autotrophicus.

In some aspects, the primer comprises a carbohydrate.

In some aspects, the carbohydrate is a sugar.

In some aspects, the sugar is fructose, glucose, sucrose, mannose, arabinose, or any combination thereof.

In some aspects, the primer is glucose.

In some aspects, the primer is fructose.

In some aspects, the sugar is sucrose.

In some aspects, the carbohydrate is molasses.

In some aspects, the primer comprises an amino acid.

In some aspects, the amino acid is glycine, glutamic acid, arginine, asparagine, tryptophan, or any combination thereof.

In some aspects, the amino acid is glutamic acid, glycine, or tryptophan.

In some aspects, the amino acid is glutamic acid, histidine, glycine, arginine, or any combination thereof.

In some aspects, the amino acid is glutamic acid.

In some aspects, the primer comprises one or more iron-rich soluble compounds.

In some aspects, the primer comprises one or more molybdenum rich-soluble compounds.

In some aspects, the molybdenum rich-soluble compound is sodium molybdate.

In some aspects, the primer comprises an organic acid.

In some aspects, the organic acid is malic acid.

In some aspects, the primer is a salt solution.

In some aspects, the salt solution is potassium chloride.

In some aspects, the primer is pantothenic acid, thiamine, or a combination thereof.

Certain aspects of the disclosure provide a method of priming nitrogen fixation, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of enhancing exopolysaccharides (EPS) production of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of enhancing microbial root colonization, comprising i) applying a primer to a biofertilizer comprising a microorganism, and ii) applying the primer and the biofertilizer to a plant, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of increasing viability of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

In some aspects, the microorganism comprises or is selected from the group consisting of Acidiphilium species, Alcaligenes species, Arthrobacter species, Azohydromonas species, Azospirillum species, Azotobacter species, Bacillus species, Beggiatoa species, Beijerinckia species, Bradyrhizobium species, Burkholderia species, Cupriavidus species, Derxia species, Herbaspirillum species, Hydrogenophaga species, Lactobacillus species, Mesorhizobium species, Methylibium species, Methylocapsa species, Methyloferula species, Methyloversatilis species, Microcyclus species, Nitrosococcus species, Nocardia species, Oligotropha species, Pannonibacter species, Paracoccus species, Pelagibaca species, Pseudomonas species, Pseudooceanicola species, Ralstonia species, Renobacter species, Rhizobium species, Rhodobacter species, Rhodomicrobium species, Rubrivivax species, Salipiger species, Sinorhizobium species, Skermanella species, Stappia species, Thauera species, Variovorax species, Xanthobacter species, and combinations thereof.

In some aspects, the microorganism comprises or is selected from the group consisting of Acidiphilium multivorum, Alcaligenes paradoxus, Azoarcus indigens, Azohydromonas australica, Azohydromonas lata, Azorhizobium caulinodans, Azospirillium brasiliense, Azospirillum amazonsense, Azospirillum lipoferum, Azospirillum lipoferum (RSAL0111), Azospirillum thiophilum, Azotobacter chroococum (MCC 0055), Azotobacter vinelandii, Azotobacter vinelandii (RSAV006), Bacillus megaterium, Bacillus pumilus, Bacillus amyloliquefaciens, Bacillus lichenformis, Bacillus subtilis, Beggiatoa alba, Beijerinckia mobilis, Bradyrhizobium elnakii, Bradyrhizobium japonicum, Bradyrhizobium japonicum (strain USDA 122), Burkholderia vietnameiensis, Cupriavidus necator, Derxia gummosa, Gluconacetobacter diazotrophicus, Gluconacetobacter diazotrophicus (MCC 0046), Herbaspirillum autrotrophicum, Herbaspirillum frisingense (MCC 0052), Hydrogenophaga pseudoflava, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Methylibium petroleiphilum, Methylocapsa aurea, Methyloferula stellate, Methyloversatilis universalis, Microcyclus aquaticus, Microcyclus ebruneus, Nitrosococcus oceani, Nitrosomonas communis, Nitrospirillum amazonense, Nocardia autotrophica, Nocardia opaca, Oligotropha carboxidovorans, Paenibacillus durus (MCC 0046), Pannonibacter phragmitetus, Paracoccus denitrificans, Paracoccus pantrophus, Paracoccus yeei, Pelagibaca bermudensis, Pseudomonas facilis, Pseudomonas fluorescens, Pseudooceanicola atlanticus, Ralstonia eutropha, Renobacter vacuolatum, Rhizobium gallicum, Rhizobium japonicum, Rhizobium japonicum (MCC 0071), Rhizobium leguminosarum, Rhizobium leguminosarum biovar viciae, Rhodobacter capsulatus, Rhodobacter sphaeroides, Rhodomicrobium vannielii, Rubrivivax gelatinosus, Salipiger mucosus, Sinorhizobium americanum, Sinorhizobium fredii, Sinorhizobium meliloti, Skermanella stibiiresistens, Stappia aggregate, Thauera humireducens, Variovorax paradoxus, Xanthobacter autotrophicus, and combinations thereof.

In some aspects, the primer is added to the biofertilizer prior to applying the biofertilizer to a plant.

In some aspects, the primer is added to the biofertilizer at the same time as applying the biofertilizer to a plant.

### BRIEF DESCRIPTION OF THE DRAWINGS/FIGURES

FIGS. 1A-1D (FIGS. 1A-1D) show quantification of X. autotrophicus N-fixation potential and transfer to lettuce. FIG. 1A (FIG. 1A) displays nitrogen fixation by X. autotrophicus in liquid culture measured with various carbon sources including fructose, glucose, citric acid, malic acid, and oxalic acid. No carbon added was used as a control. Values are reported in pg N fixed g⁻¹ dry cell weight day⁻¹±standard error (n=3). Letters indicate significant differences between treatment groups at p<0.05. FIG. 1B (FIG. 1B) displays total nitrogen fixed over the 3-day incubation per unit of carbon added. Values are reported in mg N fixed g⁻¹ C±standard error (n=3). The “No C added” treatment was not included because nitrogen fixation rates could not be normalized to g-1 C added for this treatment. Letters indicate significant differences between treatment groups at p<0.05. FIG. 1C (FIG. 1C) displays delta ¹⁵N values for leaf tissue from in planta ¹⁵N incubation experiment using lettuce grown under 75% grower standard practice (GSP) nitrogen. Values are reported in per mil±standard error (n=6). Letters indicate significant differences between treatment groups at p<0.05. FIG. 1D (FIG. 1D) displays nitrogen derived from the atmosphere (Ndfa) during a 7-day incubation measured in leaf tissue of lettuce. Values are reported in μg N±standard error (n=6). Letters indicate significant differences between treatment groups at p<0.05.

FIGS. 2A-2D (FIGS. 2A-2D) show epiphytic colonization and growth of X. autotrophicus observed in lettuce. FIG. 2A (FIG. 2A) displays a brightfield image of 5-week-old lettuce roots in glucose supplemented agar showing yellow, mucoid colonies characteristic of X. autotrophicus along root surfaces. FIG. 2B (FIG. 2B) displays a fluorescent image taken of the root using the GFP (488 nm) filter showing green fluorescing colonies of GFP-labeled X. autotrophicus along lettuce root surfaces. FIG. 2C (FIG. 2C) displays a merged image of FIG. 2A and FIG. 2B showing overlap of GFP fluorescence with colonies observed under brightfield, providing positive identification of X. autotrophicus growth on lettuce root surfaces. FIG. 2D (FIG. 2D) displays a confocal fluorescence microscope image taken of lettuce seedling roots inoculated with GFP-labeled X. autotrophicus. Root cells are stained with 0.001% CFW, while X. autotrophicus is labeled with GFP.

FIG. 3A (FIG. 3A) displays a confocal fluorescence microscope image showing epiphytic colonization of X. autotrophicus on creeping blue grass roots. Root cells are stained with 0.01% CFW. Roots were also inoculated with GFP-labeled X. autotrophicus. FIG. 3B (FIG. 3B) displays a confocal fluorescence microscope image showing epiphytic colonization of X. autotrophicus on clover roots. FIG. 3C (FIG. 3C) displays a confocal fluorescence microscope image showing epiphytic colonization of X. autotrophicus on broccoli roots. FIG. 3D (FIG. 3D) displays a confocal fluorescence microscope image showing epiphytic colonization of X. autotrophicus on tomato roots. FIG. 3E (FIG. 3E) displays a confocal fluorescence microscope image showing endophytic colonization of X. autotrophicus on tomato roots, taken after quenching root ROS using DAB and NBT stains. FIG. 3F (FIG. 3F) provides a confocal fluorescence microscope image showing endophytic colonization of X. autotrophicus on clover roots, taken after quenching root ROS using DAB and NBT stains.

FIG. 4 (FIG. 4) shows lateral root density of lettuce following treatment with X. autotrophicus/diluent (X. autotrophicus 7c grown in a bioreactor and diluted in a diluent produced using 21.1 g/L of alfalfa extract), 5% (w/v) sucrose, X. autotrophicus/diluent plus 5% (w/v) of sucrose, 5% (w/v) malic acid, X. autotrophicus/diluent plus 5% (w/v) malic acid, 5% (w/v) fructose, X. autotrophicus/diluent plus 5% (w/v) fructose, 5% (w/v) glucose, or X. autotrophicus/diluent plus 5% (w/v) glucose. Statistically significant differences are indicated by uppercase letters based on analysis of variance (ANOVA).

FIG. 5A (FIG. 5A) shows quantification of aboveground fresh biomass (g) in corn seedlings following treatment with X. autotrophicus/diluent or X. autotrophicus/diluent primed with 5% (w/v) sucrose. FIG. 5B (FIG. 5B) shows quantification of aboveground dry biomass (g) in corn seedlings following treatment with X. autotrophicus/diluent or X. autotrophicus/diluent primed with 5% (w/v) sucrose. FIG. 5C (FIG. 5C) shows leaf nitrogen (mg) quantification in corn seedlings following treatment with X. autotrophicus/diluent or X. autotrophicus/diluent primed with 5% (w/v) sucrose. Values are shown as average±standard error (n=12). Statistically significant differences are indicated by uppercase letters based on ANOVA.

FIGS. 6A-6B (FIGS. 6A-6B) show the effect of X. autotrophicus treatment on aboveground biomass in lettuce. FIG. 6A shows quantification of aboveground fresh biomass in lettuce following treatment with 80% GSP of nitrogen, X. autotrophicus/diluent, X. autotrophicus/diluent primed with 5% (w/v) sucrose, or 100% GSP of nitrogen. FIG. 6B shows quantification of aboveground dry biomass in lettuce following treatment with 80% GSP of nitrogen, X. autotrophicus/diluent, X. autotrophicus/diluent primed with 5% (w/v) sucrose, or 100% GSP of nitrogen. Aboveground fresh biomass and aboveground dry biomass are reported in grams. Values are shown as average±standard error (n=12). Statistically significant differences are indicated by uppercase letters based on ANOVA.

FIGS. 7A-7B (FIGS. 7A-7B) show the effect of X. autotrophicus treatment on leaf nitrogen content in lettuce. FIG. 7A shows leaf tissue nitrogen content (in percent w/w) of lettuce following treatment with 80% GSP of nitrogen, X. autotrophicus/diluent, X. autotrophicus/diluent primed with 5% sucrose (w/v), or 100% GSP of nitrogen. FIG. 7B shows total leaf nitrogen (in mg) of lettuce following treatment with 80% GSP of nitrogen, X. autotrophicus/diluent, X. autotrophicus/diluent primed with 5% sucrose (w/v), or 100% GSP of nitrogen. Values are shown as average±standard error (n=12). Statistically significant differences are indicated by uppercase letters based on ANOVA.

FIG. 8 (FIG. 8) shows the impact of using sucrose as a primer for X. autotrophicus/diluent. FIG. 8 shows the increase in aboveground fresh biomass compared to (1) an 80% GSP control for the following treatments: (2) 2.5% (w/v) sucrose, (3) 5% (w/v) sucrose, (4) 10% (w/v) sucrose, (5) X. autotrophicus/diluent (6) X. autotrophicus/diluent plus 2.5% (w/v) sucrose, (7) X. autotrophicus/diluent plus 5% (w/v) sucrose, (8) X. autotrophicus/diluent plus 10% (w/v) sucrose, and (9) 100% GSP. Values are shown as average±standard error (n=12). Statistically significant differences among treatments are indicated by uppercase letters based on analysis of variance (ANOVA). Asterisks indicate a treatment that was statistically significantly greater than the 80% GSP control.

FIG. 9 (FIG. 9) shows the efficacy of sucrose and malic acid as primers for freeze-dried X. autotrophicus (X. autotrophicus 7c grown in bioreactor culture and freeze-dried as a concentrated powder). FIG. 9 shows the increase in aboveground fresh compared to (1) an 80% GSP control for the following treatments: (2) freeze-dried X. autotrophicus, (3) freeze-dried X. autotrophicus plus 2.5% (w/v) sucrose, (4) freeze-dried X. autotrophicus plus 5% (w/v) sucrose, (5) freeze-dried X. autotrophicus plus 10% (w/v) sucrose, (6) freeze-dried X. autotrophicus plus 2.5% (w/v) malic acid, (7) freeze-dried X. autotrophicus plus 5% (w/v) malic acid, (8) freeze-dried X. autotrophicus plus 10% (w/v) malic acid, and (9) 100% GSP. Values are shown as average±standard error (n=12). Statistically significant differences among treatments are indicated by uppercase letters based on analysis of variance (ANOVA).

FIG. 10 (FIG. 10) shows the efficacy of malic acid, glutamic acid, and pantothenic acid as primers for freeze-dried X. autotrophicus. FIG. 10 shows the increase in aboveground fresh biomass compared to (1) an 80% GSP control for the following treatments: (2) freeze-dried X. autotrophicus, (3) 2.5% (w/v) malic acid, (4) freeze-dried X. autotrophicus plus 2.5% (w/v) malic acid, (5) 1.0 M glutamic acid, (6) freeze-dried X. autotrophicus plus 1.0 M glutamic acid, (7) 21.5 μg/mL pantothenic acid, (8) freeze-dried X. autotrophicus plus 21.5 μg/mL pantothenic acid, and (9) 100% GSP. Values are shown as average±standard error (n=12). Statistically significant differences among treatments are indicated by uppercase letters based on analysis of variance (ANOVA).

FIGS. 11A-11B (FIGS. 11A-11B) show the efficacy of food waste powders or sugars as primers for freeze-dried X. autotrophicus nitrogen fixation activity. FIG. 11A shows the rate of change in bromothymol blue absorbance over time following treatment. FIG. 11B shows the percent increase in nitrogen-fixation activity for each treatment relative to a freeze-dried X. autotrophicus control. Numbered treatment groups are as follows: (1) freeze-dried X. autotrophicus, (2) freeze-dried X. autotrophicus plus 10 mg/mL tomato powder, (3) freeze-dried X. autotrophicus plus 10 mg/mL corn powder, (4) freeze-dried X. autotrophicus plus 10 mg/mL sucrose, and (5) freeze-dried X. autotrophicus plus 10 mg/mL fructose. Values are shown as average±standard error (n=3). Statistically significant differences among treatments are indicated by uppercase letters based on analysis of variance (ANOVA).

FIGS. 12A-12B (FIGS. 12A-12B) show the efficacy of amino acids as primers for freeze-dried X. autotrophicus nitrogen-fixation activity. FIG. 12A shows the rate of change in bromothymol blue absorbance over time following treatment. FIG. 12B shows the percent increase in nitrogen-fixation activity for each treatment relative to a freeze-dried X. autotrophicus control. Numbered treatment groups are as follows: (1) freeze-dried X. autotrophicus, (2) freeze-dried X. autotrophicus plus 2.8% (w/v) arginine, (3) freeze-dried X. autotrophicus plus 0.6% (w/v) asparagine, (4) freeze-dried X. autotrophicus plus 14.6% (w/v) glutamic acid, (5) freeze-dried X. autotrophicus plus 2.3% (w/v) glycine, (6) freeze-dried X. autotrophicus plus 0.7% (w/v) tryptophan, and (7) freeze-dried X. autotrophicus plus amino acid mix (21% w/v, cumulative mixture of the individual tested amino acids). Values are shown as average±standard error (n=3). Statistically significant differences among treatments are indicated by uppercase letters based on analysis of variance (ANOVA).

FIGS. 13A-13B (FIGS. 13A-13B) show the efficacy of salt solutions and nutrient broth (NB) as primers for supporting improved rehydration recovery of freeze-dried X. autotrophicus and improved nitrogen-fixation activity. FIG. 13A shows the rate of change in bromothymol blue absorbance over time following primer treatment. FIG. 13B shows the percent increase in nitrogen-fixation activity for each treatment relative to a freeze-dried X. autotrophicus control. Numbered treatment groups are as follows: (1) freeze-dried X. autotrophicus, (2) freeze-dried X. autotrophicus plus 50 mg/mL sodium chloride, (3) freeze-dried X. autotrophicus plus 50 mg/mL potassium chloride, (4) freeze-dried X. autotrophicus plus 50 mg/mL calcium chloride, (5) freeze-dried X. autotrophicus plus 50 mg/mL sodium bicarbonate, and (6) freeze-dried X. autotrophicus plus 50 mg/mL nutrient broth. Values are shown as average±standard error (n=3). Statistically significant differences among treatments are indicated by uppercase letters based on analysis of variance (ANOVA).

FIGS. 14A-14B (FIGS. 14A-14B) show the efficacy of amino acids for use as primers of freeze-dried X. autotrophicus N-fixation activity when added during rehydration. FIG. 14A shows the rate of change in bromothyml blue absorbance over time following primer treatments. FIG. 14B shows the percent increase in nitrogen-fixation activity for each treatment relative to a freeze-dried X. autotrophicus control. Numbered treatment groups are as follows: (1) freeze-dried X. autotrophicus, (2) freeze-dried X. autotrophicus plus 0.03 M glycine, (3) freeze-dried X. autotrophicus plus 1.0 M glycine, (4) freeze-dried X. autotrophicus plus 0.03 M glutamic acid, (5) freeze-dried X. autotrophicus plus 1.0 M glutamic acid, (6) freeze-dried X. autotrophicus plus 0.03 M histidine, (7) freeze-dried X. autotrophicus plus 1.0 M histidine, (8) freeze-dried X. autotrophicus plus 0.03 M arginine, and (9) freeze-dried X. autotrophicus plus 1.0 M arginine. Values are shown as average±standard error (n=3). Statistically significant differences among treatments are indicated by uppercase letters based on analysis of variance (ANOVA).

FIGS. 15A-15B (FIGS. 15A-15B) show the efficacy of thiamine and pantothenic acid as primers for N-fixation activity of freeze-dried X. autotrophicus. FIG. 15A shows the rate of change in bromothymol blue absorbance over time following primer treatments. FIG. 15B shows the percent increase in nitrogen-fixation activity for each treatment relative to a freeze-dried X. autotrophicus control. Numbered treatment groups are as follows: (1) freeze-dried X. autotrophicus, (2) freeze-dried X. autotrophicus plus 0.3 μg/mL thiamine, (3) freeze-dried X. autotrophicus plus 0.7 μg/mL thiamine, (4) freeze-dried X. autotrophicus plus 3.3 μg/mL thiamine, (5) freeze-dried X. autotrophicus plus 6.7 μg/mL thiamine, (6) freeze-dried X. autotrophicus plus 2.2 μg/mL pantothenic acid, (7) freeze-dried X. autotrophicus plus 4.3 μg/mL pantothenic acid, (8) freeze-dried X. autotrophicus plus 21.5 μg/mL pantothenic acid, (9) freeze-dried X. autotrophicus plus 43.0 μg/mL pantothenic acid, and (10) freeze-dried X. autotrophicus plus 0.7 μg/mL thiamine and 4.3 μg/mL pantothenic acid. Values are shown as average±standard error (n=3). Statistically significant differences among treatments are indicated by uppercase letters based on analysis of variance (ANOVA).

FIGS. 16A-16D (FIGS. 16A-16D) show the efficacy of molybdenum (sodium molybdate) and iron (iron ethylenediaminetetraacetic acid (EDTA)) as primers for X. autotrophicus/diluent. FIGS. 16A and 16C shows the rate of change in bromothymol blue absorbance over time following primer treatments. FIGS. 16B and 16D shows the percent increase in nitrogen-fixation activity for each treatment relative to a X. autotrophicus/diluent control. Numbered treatment groups are as follows: (1) X. autotrophicus/diluent, (2) X. autotrophicus/diluent plus sodium molybdate at 2x concentration (0.016 mM), (3) X. autotrophicus/diluent plus sodium molybdate at 5x concentration (0.064 mM), (4) X. autotrophicus/diluent plus sodium molybdate at 1Ox concentration (0.080 mM), (5) X. autotrophicus/diluent plus iron EDTA at 2x concentration (0.358 mM), (6) X. autotrophicus/diluent plus iron EDTA at 5x concentration (0.895 mM), and (7) X. autotrophicus/diluent plus iron EDTA at 1Ox concentration (1.790 mM). Values are shown as average±standard error (n=3). Statistically significant differences among treatments are indicated by uppercase letters based on analysis of variance (ANOVA).

FIGS. 17A-17B (FIGS. 17A-17B) show the efficacy of molasses as a primer for the N-fixation activity of freeze-dried X. autotrophicus. FIG. 17A shows the rate of change in bromothymol blue absorbance over time following primer treatments. FIG. 17B shows the percent increase in nitrogen-fixation activity for each treatment relative to a freeze-dried X. autotrophicus control. Numbered treatment groups are as follows: (1) freeze-dried X. autotrophicus, (2) freeze-dried X. autotrophicus plus 0.5 g/L molasses, (3) freeze-dried X. autotrophicus plus 1 g/L molasses, (4) freeze-dried X. autotrophicus plus 2.0 g/L molasses, (5) freeze-dried X. autotrophicus plus 4.0 g/L molasses, and (6) freeze-dried X. autotrophicus plus 8.0 g/L molasses. Values are shown as average±standard error (n=3). Statistically significant differences among treatments are indicated by uppercase letters based on analysis of variance (ANOVA).

### DETAILED DESCRIPTION

Certain aspects of the disclosure provide a composition comprising i) a biofertilizer comprising a microorganism, wherein the microorganism is Xanthobacter autotrophicus, and ii) a primer of nitrogen fixation, wherein the primer is glucose, fructose, sucrose, malic acid, pantothenic acid, thiamine, an amino acid, potassium chloride, sodium molybdate, molasses, or any combination thereof.

Certain aspects of the disclosure provide a composition comprising i) a biofertilizer comprising a microorganism, wherein the microorganism is Xanthobacter autotrophicus, and ii) a primer of nitrogen fixation.

Certain aspects of the disclosure provide a composition comprising i) a biofertilizer comprising a microorganism, and ii) a primer of nitrogen fixation, wherein the primer is fructose glucose, sucrose, malic acid, pantothenic acid, thiamine, an amino acid, potassium chloride, sodium molybdate, molasses, or any combination thereof.

Certain aspects of the disclosure provide a kit comprising any of the compositions disclosed herein.

Certain aspects of the disclosure provide a kit comprising i) a biofertilizer comprising a microorganism and ii) a primer of nitrogen fixation.

Certain aspects of the disclosure provide a method of priming nitrogen fixation, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof, and wherein the microorganism is Xanthobacter autotrophicus.

Certain aspects of the disclosure provide a method of enhancing exopolysaccharides (EPS) production of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof, and wherein the microorganism is Xanthobacter autotrophicus.

Certain aspects of the disclosure provide a method of enhancing microbial root colonization of a plant, comprising i) applying a primer to a biofertilizer comprising a microorganism, and ii) applying the primer and the biofertilizer to a plant, wherein the primer comprises glucose, fructose, sucrose, malic acid, pantothenic acid, thiamine, an amino acid, potassium chloride, sodium molybdate, molasses, or a combination thereof, and wherein the microorganism is Xanthobacter autotrophicus.

Certain aspects of the disclosure provide a method of increasing viability of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof, and wherein the microorganism is Xanthobacter autotrophicus.

Certain aspects of the disclosure provide a method of priming nitrogen fixation, comprising applying a primer to a biofertilizer comprising a microorganism, wherein the microorganism is Xanthobacter autotrophicus.

Certain aspects of the disclosure provide a method of enhancing exopolysaccharides (EPS) production of a biofertilizer, comprising applying a primer to a biofertilizer comprising a microorganism, wherein the microorganism is Xanthobacter autotrophicus.

Certain aspects of the disclosure provide a method of enhancing microbial root colonization, comprising i) applying a primer to a biofertilizer comprising a microorganism, and ii) applying the primer and the biofertilizer to a plant, wherein the microorganism is Xanthobacter autotrophicus.

Certain aspects of the disclosure provide a method of increasing viability of a biofertilizer, comprising applying a primer to a biofertilizer comprising a microorganism, wherein the microorganism is Xanthobacter autotrophicus.

Certain aspects of the disclosure provide a method of priming nitrogen fixation, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of enhancing exopolysaccharides (EPS) production of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of enhancing microbial root colonization, comprising i) applying a primer to a biofertilizer comprising a microorganism, and ii) applying the primer and the biofertilizer to a plant, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of increasing viability of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference. Incorporation by reference of any such documents shall not be considered an admission that the incorporated materials are prior art to the present disclosure, or considered as material to the patentability of the present disclosure.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean about, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this application, various embodiments of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6 should be considered to have specifically disclosed subranges such as from 1 to 2, from 1 to 3, from 1 to 4, from 1 to 5, from 2 to 3, from 2 to 4, from 2 to 5, from 2 to 6, from 3 to 4, from 3 to 5, from 3 to 6, etc., as well as individual numbers within that range, for example, 1, 2, 3, 4, 5, and 6. This applies regardless of the breadth of the range.

The terms “comprises,” “comprising,” “includes,” “including,” “having,” and their conjugates as used herein are interchangeable and mean “including but not limited to.” It is understood that wherever aspects are described herein with the language “comprising,” otherwise analogous aspects described in terms of “consisting of” and/or “consisting essentially of” are also provided.

The term “consisting of” as used herein means including and limited to.

The term “consisting essentially of” as used herein means the specified material of a composition, or the specified steps of a method, and those additional materials or steps that do not materially affect the basic characteristics of the material or method.

As used herein, the term “effective amount” in terms of a biofertilizer will depend upon a variety of factors, including, for example, percent viability of cells in the biofertilizer, concentration of cells in the biofertilizer, the levels of nutrients, including ammonia and carbon sources (e.g., PHB), and whether the biofertilizer is in the form of a liquid cell suspension or comprises a solid biomass component, such as soils, plant materials, or inert materials. A person of ordinary skill in the art will be able to determine an effective amount taking into account these variables. For purposes of the instant disclosure, an effective amount of a biofertilizer means an amount of the biofertilizer that is sufficient to result in an enhanced property or characteristic of a soil microbiome and/or a crop or plant that is statistically greater than the same property or characteristic in the absence of the biofertilizer, such as, for example, increased crop yield, increased fruit or vegetable yield or root storage mass, increased carbon and/or nitrogen availability in the microbiome. In some aspects, the property or characteristic (e.g., crop or plant yield or yield quality) enhanced by the biofertilizer is observed with at least a 5%, or at least a 6%, or 7%, or 8%, or 9%, or 10%, or 25%, or 50%, or 75%, or 100%, or 200%, or 300%, or 400%, or 500%, or 1000%, or 1250%, or 1500%, or 2000%, or more increase over the same property or characteristic established in the absence of the biofertilizer.

As used herein, the term “microbiome” refers to the collection of all microorganisms living in a particular environment, including in the soil surrounding and/or interacting with the root of a plant.

As used herein, the term “biofertilizer” refers to a preparation containing living cells or latent cells of microorganisms that help plants (e.g., crop plants) grow. The term also refers to a preparation containing living cells or latent cells of microorganisms that help to feed and/or enhance the soil microbiome. The term also refers to a preparation containing living cells or latent cells of microorganisms that produce chemicals (including but not limited to nitrate, ammonia, phosphorus) to directly or indirectly provide nutrition to plants (e.g., crop plants), or to directly or indirectly signal plant or microbial pathways to the benefit of the plant (e.g. crop plants), in the soil, soilless substrate, or other growth medium. In some aspects, the biofertilizer can be a preparation containing living cells or latent cells of microorganisms, or having enhanced accumulation of microbial intracellular storage compound (MISC), or a combination thereof. In some aspects, the MISC accumulated by the microorganism comprises a polyhydroxyalkanoate (PHA), a polyphosphate (PolyP), a lipid, or a combination thereof. In some aspects, the MISC is a PHA. In some aspects, the PHA is polyhydroxybutyrate (PHB), poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In one aspect, the PHA is PHB.

As used herein, the term “nitrogenase” refers to an enzyme that is produced by certain specialized bacteria called nitrogen-fixing bacteria, such as cyanobacteria and Xanthobacter (e.g., X. autotrophicus), which are responsible for reducing atmospheric nitrogen (N₂) to ammonia (NH₃) as part of the nitrogen cycle.

The term “derived from” as used herein refers to a component that is isolated from or made using a plant (e.g., an extract or tea), yeast (e.g. spent brewing yeast), insect (e.g., honey), or algae. For example, a diluent that is derived from a plant can include a tea obtained from plant biomass.

The term “primer” as used herein refers to a compound that increases an activity of a microorganism (e.g., Xanthobacter autotrophicus) at the time of application to a plant and/or soil. In some aspects, the primer is combined with the microorganism in advance of application to the plant and/or soil. In some aspects, the primer is combined with the microorganism at the time of application to the plant and/or soil. In some aspects, the ability increased by the primer is the ability of the microorganism to fix nitrogen. In some aspects, the ability increased by the primer is the ability of the microorganism to adhere to and colonize root surfaces. In some aspects, the ability increased by the primer is the viability of the microorganism.

Compositions and/or Formulations

Certain aspects of the disclosure provide a composition comprising i) a biofertilizer comprising a microorganism, wherein the microorganism is Xanthobacter autotrophicus, and ii) a primer of nitrogen fixation, wherein the primer is glucose, fructose, or a combination thereof.

Certain aspects of the disclosure provide a composition comprising i) a biofertilizer comprising a microorganism, wherein the microorganism is Xanthobacter autotrophicus, and ii) a primer of nitrogen fixation.

Certain aspects of the disclosure provide a composition comprising i) a biofertilizer comprising a microorganism, and ii) a primer of nitrogen fixation, wherein the primer is fructose or glucose.

Certain aspects of the disclosure provide a composition comprising i) a biofertilizer comprising a microorganism, wherein the microorganism is Xanthobacter autotrophicus, and ii) a primer of nitrogen fixation, wherein the primer is glucose, fructose, sucrose, malic acid, pantothenic acid, thiamine, an amino acid, potassium chloride, sodium molybdate, molasses, or a combination thereof.

Certain aspects of the disclosure provide a composition comprising i) a biofertilizer comprising a microorganism, and ii) a primer of nitrogen fixation, wherein the primer is fructose, glucose, sucrose, malic acid, pantothenic acid, thiamine, an amino acid, potassium chloride, sodium molybdate, molasses, or a combination thereof.

In some aspects, the primer comprises a carbohydrate.

In some aspects, the carbohydrate is a sugar. In some aspects, the primer comprises a source of the carbohydrate (e.g., molasses).

In some aspects, the sugar is sucrose, mannose, arabinose, glucose, fructose, or combinations thereof.

In some aspects, the primer is glucose.

In some aspects, the primer is fructose.

In some aspects, the sugar is sucrose.

In some aspects, the carbohydrate is molasses.

In some aspects, the primer comprises an amino acid.

In some aspects, the amino acid is glycine.

In some aspects, the amino acid is glycine, glutamic acid, arginine, asparagine, tryptophan, or any combination thereof

In some aspects, the amino acid is glutamic acid, glycine, or tryptophan.

In some aspects, the amino acid is glutamic acid, histidine, glycine, arginine, or any combination thereof.

In some aspects, the primer comprises one or more iron-rich soluble compounds.

In some aspects, the primer comprises one or more molybdenum rich-soluble compounds.

In some aspects, the molybdenum rich-soluble compound is sodium molybdate.

In some aspects, the primer comprises an organic acid.

In some aspects, the organic acid is malic acid.

In some aspects, the primer is a salt solution.

In some aspects, the salt solution is potassium chloride.

In some aspects, the primer is pantothenic acid, thiamine, or a combination thereof.

In some aspects, the primer comprises a source of glycine.

In some aspects, the primer comprises a source of one or more amino acids.

In some aspects, the primer comprises a source of one or more iron-rich soluble compounds.

In some aspects, the primer comprises a source of one or more molybdenum rich-soluble compounds.

In some aspects, the primer is glucose.

In some aspects, the primer is fructose.

In some aspects, the primer is not citric acid, malic acid, or oxalic acid.

In some aspects, the microorganism is a nitrogen fixing microorganism.

In some aspects, the nitrogen fixing microorganism is a bacteria.

In some aspects, the nitrogen-fixing microorganism expresses nitrogenase. In some aspects, the nitrogen-fixing microorganism accumulates a microbial intracellular storage compound (MISC). In some aspects, the nitrogen-fixing microorganism expresses nitrogenase and accumulates a MISC. In some aspects, the MISC comprises a polyhydroxyalkanoate (PHA), a polyphosphate (PolyP), or a lipid, or a combination thereof. In some aspects, the MISC is a PHA. In some aspects, the PHA is polyhydroxybutyrate (PHB) poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In one aspect, the PHA is PHB.

In some aspects, the nitrogen-fixing microorganism in the biofertilizer comprises bacteria. In some aspects, the nitrogen-fixing microorganism is a PHA-producing bacteria. In some aspects, the nitrogen-fixing microorganism is a PHB-producing bacteria. In some aspects, the nitrogen-fixing microorganism is a PHV-producing bacteria.

In some aspects, the microorganism comprises an MISC accumulation pathway.

In some aspects, the microorganism comprises or is selected from the group consisting of Acidiphilium species, Alcaligenes species, Arthrobacter species, Azohydromonas species, Azospirillum species, Azotobacter species, Bacillus species, Beggiatoa species, Beijerinckia species, Bradyrhizobium species, Burkholderia species, Cupriavidus species, Derxia species, Herbaspirillum species, Hydrogenophaga species, Lactobacillus species, Mesorhizobium species, Methylibium species, Methylocapsa species, Methyloferula species, Methyloversatilis species, Microcyclus species, Nitrosococcus species, Nocardia species, Oligotropha species, Pannonibacter species, Paracoccus species, Pelagibaca species, Pseudomonas species, Pseudooceanicola species, Ralstonia species, Renobacter species, Rhizobium species, Rhodobacter species, Rhodomicrobium species, Rubrivivax species, Salipiger species, Sinorhizobium species, Skermanella species, Stappia species, Thauera species, Variovorax species, Xanthobacter species, and combinations thereof.

In some aspects, the microorganism comprises or is selected from the group consisting of Acidiphilium multivorum, Alcaligenes paradoxus, Azoarcus indigens, Azohydromonas australica, Azohydromonas lata, Azorhizobium caulinodans, Azospirillium brasiliense, Azospirillum amazonsense, Azospirillum lipoferum, Azospirillum lipoferum (RSAL0111), Azospirillum thiophilum, Azotobacter chroococum (MCC 0055), Azotobacter vinelandii, Azotobacter vinelandii (RSAV006), Bacillus megaterium, Bacillus pumilus, Bacillus amyloliquefaciens, Bacillus lichenformis, Bacillus subtilis, Beggiatoa alba, Beijerinckia mobilis, Bradyrhizobium elnakii, Bradyrhizobium japonicum, Bradyrhizobium japonicum (strain USDA 122), Burkholderia vietnameiensis, Cupriavidus necator, Derxia gummosa, Gluconacetobacter diazotrophicus, Gluconacetobacter diazotrophicus (MCC 0046), Herbaspirillum autrotrophicum, Herbaspirillum frisingense (MCC 0052), Hydrogenophaga pseudoflava, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Methylibium petroleiphilum, Methylocapsa aurea, Methyloferula stellate, Methyloversatilis universalis, Microcyclus aquaticus, Microcyclus ebruneus, Nitrosococcus oceani, Nitrosomonas communis, Nitrospirillum amazonense, Nocardia autotrophica, Nocardia opaca, Oligotropha carboxidovorans, Paenibacillus durus (MCC 0046), Pannonibacter phragmitetus, Paracoccus denitrificans, Paracoccus pantrophus, Paracoccus yeei, Pelagibaca bermudensis, Pseudomonas facilis, Pseudomonas fluorescens, Pseudooceanicola atlanticus, Ralstonia eutropha, Renobacter vacuolatum, Rhizobium gallicum, Rhizobium japonicum, Rhizobium japonicum (MCC 0071), Rhizobium leguminosarum, Rhizobium leguminosarum biovar viciae, Rhodobacter capsulatus, Rhodobacter sphaeroides, Rhodomicrobium vannielii, Rubrivivax gelatinosus, Salipiger mucosus, Sinorhizobium americanum, Sinorhizobium fredii, Sinorhizobium meliloti, Skermanella stibiiresistens, Stappia aggregate, Thauera humireducens, Variovorax paradoxus, Xanthobacter autotrophicus, and combinations thereof.

In some aspects, the microorganism is Xanthobacter autotrophicus.

In some aspects, the Xanthobacter autotrophicus comprises Xanthobacter autotrophicus DSM 431, Xanthobacter autotrophicus DSM 432, Xanthobacter autotrophicus DSM 597, Xanthobacter autotrophicus DSM 685, Xanthobacter autotrophicus DSM 1393, Xanthobacter autotrophicus DSM 1618, Xanthobacter autotrophicus DSM 2009, Xanthobacter autotrophicus DSM 2267, Xanthobacter autotrophicus DSM 3874, Xanthobacter autotrophicus CCUG 44692, or any other Xanthobacter autotrophicus strain associated with NCBI Taxonomy ID 280.

In some aspects, the biofertilizer is in liquid formulation.

In some aspects, the biofertilizer is in a dry formulation.

In some aspects, the primer is in liquid formulation.

In some aspects, the primer is in a dry formulation. In some aspects, the primer is a powder.

In some aspects, the microorganism is in a powder.

In some aspects, the microorganism is lyophilized.

In some aspects, the microorganism is freeze-dried. Freeze-drying involves, for example, the removal of liquid from the microorganism.

In some aspects, the microorganism has been dried by spray drying. Spray drying generally produces small liquid droplets of specific sizes from a liquid or slurry using a spray nozzle or atomizer. After the droplets exit the nozzle or atomizer, they are dried, generally using hot air, to form a powder. Machines known as spray dryers are normally used for this process.

Primers or microorganisms (generally liquid, but also semi-solid or solid) may be dried, dehydrated or desiccated using a variety of methods. In some examples, a liquid sample may be left open so that moisture from the sample is evaporated into the air. This may be called air drying. In some examples, a gas stream (e.g., air) may apply heat to the sample by convection and moisture/vapor is removed as humidity. In some aspects, vacuum drying, where heat is supplied to the sample by conduction, radiation, or microwaves, vapor is produced and carried away by a vacuum system, may be used. In some aspects, drum drying, where a surface supplies heat to the sample, vapor is produced and carried away by an aspirator, may be used. In some aspects, a dried sample may be produced by draining (e.g., centrifugation to mechanically extract a solvent).

In some aspects, both the primer and microorganisms are in a dry formulation (e.g., both before being combined to form a composition, and within the composition).

In some aspects, both the primer and microorganisms are in a liquid formulation (e.g., both before being combined to form a composition, and within the composition).

In some aspects, the primer is in a liquid formulation and the microorganisms are in a dry formulation prior to being combined to form a composition.

In some aspects, the primer is in a dry formulation and the microorganisms are in a liquid formulation prior to being combined to form a composition.

In some aspects, the liquid formulation is concentrated to remove water.

In some aspects, the composition comprises about 50 μg to about 150 μg of primer per 1 mL of the biofertilizer.

In some aspects, the composition comprises about 50 μg to about 150 μg, about 75 μg to about 150 μg, about 100 μg to about 150 μg, about 125 μg to about 150 μg, about 50 μg to about 125 μg, about 50 μg to about 100 μg, about 50 μg to about 75 μg, about 75 μg to about 125 μg, about 75 μg to about 100 μg, or about 100 μg to about 125 μg of primer per 1 mL of the biofertilizer.

In some aspects, the composition comprises about 50 μg, about 75 μg, about 100 μg, about 125 μg, or about 150 μg of primer per 1 mL of the biofertilizer.

In some aspects, the composition comprises about 100 μg of primer per 1 mL of the biofertilizer.

In some aspects, the composition comprises about 0.05 g to about 10 g of primer per 1 L of the biofertilizer.

In some aspects, the composition comprises about 0.05 g to about 0.15 g, about 0.075 g to about 0.15 g, about 0.1 g to about 0.15 g, about 0.125 g to about 0.15 g, about 0.05 g to about 0.125 g, about 0.05 g to about 0.1 g, about 0.05 g to about 0.075 g, about 0.075 g to about 0.125 g, about 0.1 g to about 0.125 g, about 0.075 g to about 0.1 g, about 0.05 g to about 0.5 g, about 0.05 g to about 1 g, about 0.05 g to about 1.5 g, about 0.05 g to about 2 g, about 0.05 g to about 2.5 g, about 0.05 g to about 3 g, about 0.05 g to about 4 g, about 0.05 g to about 5 g, about 0.05 g to about 6 g, about 0.05 g to about 7 g, about 0.05 g to about 8 g, about 0.05 g to about 9 g, or about 0.05 g to about 10 g of primer per 1 L of the biofertilizer.

In some aspects, the composition comprises about 0.05 g, about 0.075 g, about 0.1 g, about 0.125 g, about 0.15 g, about 0.25 g, about 0.5 g, about 1 g, about 1.5 g, about 2 g, about 2.5 g, about 3 g, about 4 g, about 5 g, about 6 g, about 7 g, about 8 g, about 9 g, or about 10 g of primer per 1 L of the biofertilizer.

In some aspects, the composition comprises about 0.1 g of primer per 1 L of the biofertilizer.

In some aspects, the composition comprises about 5% w/v of the primer.

In some aspects, the composition comprises about 1% w/v to about 6% w/v, about 1% w/v to about 5% w/v, about 1% w/v to about 4% w/v, about 1% w/v to about 3% w/v, about 1% w/v to about 2% w/v, about 2% w/v to about 6% w/v, about 3% w/v to about 6% w/v, about 4% w/v to about 6% w/v, about 2% w/v to about 5% w/v, about 2% w/v to about 4% w/v, about 2% w/v to about 3% w/v, about 3% w/v to about 5% w/v, or about 3% w/v to about 4% w/v of the primer.

In some aspects, the composition comprises about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, or about 6% w/v of the primer.

In some aspects, the composition comprises about 2.5% w/v of the primer.

In some aspects, the composition comprises about 21.5 μg/mL of the primer.

In some aspects, the composition comprises about 15 μg/mL to about 25 μg/mL, 15 μg/mL to about 20 μg/mL, 20 μg/mL to about 25 μg/mL, 21.5 μg/mL to about 25 μg/mL, 15 μg/mL to about 21.5 μg/mL, 17.5 μg/mL to about 21.5 μg/mL, or 21.5 μg/mL to about 23 μg/mL of the primer.

In some aspects, the composition comprises about 15 μg/mL, 17.5 μg/mL, 21.5 μg/mL, about 23 μg/mL, or about 25 μg/mL of the primer.

In some aspects, the amino acid is arginine.

In some aspects, the composition comprises about 2% w/v to about 3% w/v of the primer, optionally about 2.8% w/v of the primer.

In some aspects, the composition comprises about 2.8% w/v of the primer.

In some aspects, the amino acid is asparagine.

In some aspects, the composition comprises about 0.5% w/v to about 1% w/v of the primer, optionally about 0.6% w/v of the primer.

In some aspects, the composition comprises about 0.5% w/v to about 2% w/v, about 1% w/v to about 2% w/v, about 1.50% w/v to about 2% w/v, about 0.50% w/v to about 1.5% w/v, or about 0.5% w/v to about 1% w/v of the primer.

In some aspects, the composition comprises about 0.5% w/v, about 1% w/v, about 1.5% w/v, or about 2% w/v of the primer.

In some aspects, the composition comprises about 0.6% w/v of the primer.

In some aspects, the amino acid is glutamic acid.

In some aspects, the composition comprises about 10% w/v to about 20% w/v of the primer, optionally about 14.6% w/v of the primer.

In some aspects, the composition comprises about 10% w/v to about 25% w/v, about 10% w/v to about 22.5% w/v, about 10% w/v to about 20% w/v, about 10% w/v to about 17.5% w/v, about 10% w/v to about 15% w/v, about 10% w/v to about 12.5% w/v, about 12.5% w/v to about 20% w/v, about 15% w/v to about 20% w/v, about 17.5% w/v to about 20% w/v, about 17.5% w/v to about 25% w/v, or about 20% w/v to about 25% w/v of the primer.

In some aspects, the composition comprises about 10% w/v, about 12.5% w/v, about 15% w/v, about 17.5% w/v, about 20% w/v, or about 25% w/v of the primer.

In some aspects, the composition comprises about 14.6% w/v of the primer.

In some aspects, the amino acid is glycine.

In some aspects, the composition comprises about 1.5% w/v to about 3% w/v of the primer, optionally about 2.3% w/v of the primer.

In some aspects, the composition comprises about 2.3% w/v of the primer.

In some aspects, the amino acid is tryptophan.

In some aspects, the composition comprises about 0.5% w/v to about 1% w/v of the primer, optionally about 0.7% w/v of the primer.

In some aspects, the composition comprises about 0.7% w/v of the primer.

In some aspects, the amino acid is a combination of the amino acids.

In some aspects, the composition comprises about 15% w/v to about 25% w/v of the primer, optionally about 21% w/v of the primer.

In some aspects, the composition comprises about 21% w/v of the primer.

In some aspects, the composition comprises about 25 mg/mL to about 75 mg/mL of the primer, optionally about 50 mg/mL of the primer.

In some aspects, the composition comprises about 25 mg/mL to about 75 mg/mL, about 25 mg/mL to about 65 mg/mL, about 25 mg/mL to about 55 mg/mL, about 25 mg/mL to about 50 mg/mL, about 25 mg/mL to about 45 mg/mL, about 25 mg/mL to about 35 mg/mL, about 35 mg/mL to about 75 mg/mL, about 45 mg/mL to about 75 mg/mL, about 50 mg/mL to about 75 mg/mL, about 55 mg/mL to about 75 mg/mL, about 65 mg/mL to about 75 mg/mL, about 35 mg/mL to about 50 mg/mL, or about 50 mg/mL to about 65 mg/mL of the primer.

In some aspects, the composition comprises about 25 mg/mL, about 35 mg/mL, about 45 mg/mL, about 50 mg/mL, about 55 mg/mL, about 65 mg/mL, or about 75 mg/mL of the primer.

In some aspects, the composition comprises about 50 mg/mL of the primer.

In some aspects, the composition comprises about 0.025 M to about 0.05 M of the primer, optionally about 0.03 M of the primer.

In some aspects, the composition comprises about 0.5 M to about 1.5 M of the primer, optionally about 1.0 M of the primer.

In some aspects, the composition comprises about 0.5 M to about 1.5 M, about 1.0 M to about 1.5 M, or about 0.5 M to about 1.0 M of the primer.

In some aspects, the composition comprises about 0.5 M, about 1.0 M, or about 1.5 M of the primer.

In some aspects, the composition comprises about 0.03 M of the primer.

In some aspects, the composition comprises about 1.0 M of the primer.

In some aspects, the primer is thiamine.

In some aspects, the composition comprises about 3 μg/mL to about 4 μg/mL of the primer, optionally about 3.3 μg/mL of the primer.

In some aspects, the composition comprises about 2 μg/mL to about 6 μg/mL, about 3 μg/mL to about 6 μg/mL, about 4 μg/mL to about 6 μg/mL, about 5 μg/mL to about 6 μg/mL, about 2 μg/mL to about 5 μg/mL, about 2 μg/mL to about 4 μg/mL, about 2 μg/mL to about 3 μg/mL, about 3 μg/mL to about 4 μg/mL, about 3.5 μg/mL to about 4 g/mL, or about 3 μg/mL to about 3.5 μg/mL of the primer.

In some aspects, the composition comprises about 2 μg/mL, about 3 μg/mL, about 3.5 μg/mL, about 4 μg/mL, about 5 μg/mL, or about 6 μg/mL of the primer.

In some aspects, the composition about 3.3 μg/mL of the primer.

In some aspects, the composition comprises about 6 μg/mL to about 8 μg/mL of the primer, optionally about 6.7 μg/mL of the primer.

In some aspects, the composition comprises about 6 μg/mL to about 8 μg/mL, about 6 μg/mL to about 7 μg/mL, or about 7 μg/mL to about 8 μg/mL of the primer.

In some aspects, the composition comprises about 6 μg/mL, about 7 μg/mL, or about 8 μg/mL of the primer.

In some aspects, the composition about 6.7 μg/mL of the primer.

In some aspects, the primer is pantothenic acid.

In some aspects, the composition comprises about 2 μg/mL to about 3 μg/mL of the primer, optionally about 2.2 μg/mL of the primer.

In some aspects, the composition comprises about 3 μg/mL to about 5 μg/mL of the primer, optionally about 4.3 μg/mL of the primer.

In some aspects, the composition comprises about 20 μg/mL to about 25 μg/mL of the primer, optionally about 21.5 μg/mL of the primer.

In some aspects, the composition comprises about 20 μg/mL to about 25 μg/mL, about 15 μg/mL to about 30 μg/mL, about 15 μg/mL to about 25 μg/mL, about 15 g/mL to about 20 μg/mL, about 20 μg/mL to about 30 μg/mL, about 25 μg/mL to about 30 g/mL or about 15 μg/mL to about 25 μg/mL of the primer.

In some aspects, the composition comprises about 15 μg/mL, about 20 μg/mL, about 25 μg/mL, or about 30 μg/mL of the primer.

In some aspects, the composition comprises about 2.2 μg/mL of the primer.

In some aspects, the composition comprises about 4.3 μg/mL of the primer.

In some aspects, the composition comprises about 21.5 μg/mL of the primer.

In some aspects, the primer is a combination of thiamine and pantothenic acid.

In some aspects, the composition comprises about 0.7 μg/mL of thiamine and about 4.3 μg/mL of pantothenic acid.

In some aspects, the composition comprises about 0.5 μg/mL to about 1.0 μg/mL, about 0.75 μg/mL to about 1.0 μg/mL, or about 0.5 μg/mL to about 0.75 μg/mL of thiamine.

In some aspects, the composition comprises about 0.5 μg/mL, about 0.75 μg/mL, or about 1.0 μg/mL of thiamine.

In some aspects, the composition comprises about 4 μg/mL to about 6 μg/mL, about 5 μg/mL to about 6 μg/mL, or about 4 μg/mL to about 5 μg/mL of pantothenic acid.

In some aspects, the composition comprises about 4 μg/mL, about 5 μg/mL, or about 6 μg/mL of pantothenic acid.

In some aspects, the composition comprises about 0.010 mM to about 0.1 mM of the primer, optionally about 0.016 mM, about 0.064 mM, or about 0.080 mM of the primer.

In some aspects, the composition comprises about 0.010 mM to about 0.1 mM, about 0.010 mM to about 0.070 mM, about 0.010 mM to about 0.050 mM, about 0.010 mM to about 0.030 mM, about 0.030 mM to about 0.1 mM, about 0.050 mM to about 0.1 mM, or about 0.070 mM to about 0.1 mM of the primer.

In some aspects, the composition comprises about 0.010 mM, about 0.030 mM, about 0.050 mM, about 0.070 mM, or about 0.1 mM of the primer.

In some aspects, the composition comprises about 1.0 g/L or about 4.0 g/L of the primer.

In some aspects, the composition comprises about 1.0 g/L of the primer.

In some aspects, the composition comprises about 4.0 g/L of the primer.

### Kits

Certain aspects of the disclosure provide a kit comprising any of the compositions disclosed herein.

Certain aspects of the disclosure provide a kit comprising i) a biofertilizer comprising a microorganism and ii) a primer of nitrogen fixation.

In some aspects, the primer comprises a carbohydrate.

In some aspects, the carbohydrate is a sugar.

In some aspects, the sugar is fructose, glucose, sucrose, mannose, arabinose, or any combination thereof.

In some aspects, the primer is glucose.

In some aspects, the primer is fructose.

In some aspects, the sugar is sucrose.

In some aspects, the carbohydrate is molasses.

In some aspects, the primer comprises an amino acid.

In some aspects, the amino acid is glycine, glutamic acid, arginine, asparagine, tryptophan, or any combination thereof.

In some aspects, the amino acid is glutamic acid, glycine, or tryptophan.

In some aspects, the amino acid is glutamic acid, histidine, glycine, arginine, or any combination thereof.

In some aspects, the primer comprises one or more iron-rich soluble compounds.

In some aspects, the primer comprises one or more molybdenum rich-soluble compounds.

In some aspects, the molybdenum rich-soluble compound is sodium molybdate.

In some aspects, the primer comprises an organic acid.

In some aspects, the organic acid is malic acid.

In some aspects, the primer is a salt solution.

In some aspects, the salt solution is potassium chloride.

In some aspects, the primer is pantothenic acid, thiamine, or a combination thereof.

In some aspects, the primer comprises a source of glycine.

In some aspects, the primer comprises a source of one or more amino acids.

In some aspects, the primer comprises a source of one or more iron-rich soluble compounds.

In some aspects, the primer comprises a source of one or more molybdenum rich-soluble compounds.

In some aspects, the primer is not citric acid, malic acid, or oxalic acid.

In some aspects, the microorganism is a nitrogen fixing microorganism.

In some aspects, the nitrogen fixing microorganism is a bacteria.

In some aspects, the nitrogen-fixing microorganism expresses nitrogenase. In some aspects, the nitrogen-fixing microorganism accumulates a microbial intracellular storage compound (MISC). In some aspects, the nitrogen-fixing microorganism expresses nitrogenase and accumulates a MISC. In some aspects, the MISC comprises a polyhydroxyalkanoate (PHA), a polyphosphate (PolyP), or a lipid, or a combination thereof. In some aspects, the MISC is a PHA. In some aspects, the PHA is polyhydroxybutyrate (PHB) poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In one aspect, the PHA is PHB.

In some aspects, the nitrogen-fixing microorganism in the biofertilizer comprises bacteria. In some aspects, the nitrogen-fixing microorganism is a PHA-producing bacteria. In some aspects, the nitrogen-fixing microorganism is a PHB-producing bacteria. In some aspects, the nitrogen-fixing microorganism is a PHV-producing bacteria.

In some aspects, the microorganism comprises an MISC accumulation pathway.

In some aspects, the microorganism is Xanthobacter autotrophicus.

In some aspects, the Xanthobacter autotrophicus comprises Xanthobacter autotrophicus DSM 431, Xanthobacter autotrophicus DSM 432, Xanthobacter autotrophicus DSM 597, Xanthobacter autotrophicus DSM 685, Xanthobacter autotrophicus DSM 1393, Xanthobacter autotrophicus DSM 1618, Xanthobacter autotrophicus DSM 2009, Xanthobacter autotrophicus DSM 2267, Xanthobacter autotrophicus DSM 3874, Xanthobacter autotrophicus CCUG 44692, or any other Xanthobacter autotrophicus strain associated with NCBI Taxonomy ID 280.

In some aspects, the microorganism comprises or is selected from the group consisting of Acidiphilium species, Alcaligenes species, Arthrobacter species, Azohydromonas species, Azospirillum species, Azotobacter species, Bacillus species, Beggiatoa species, Beijerinckia species, Bradyrhizobium species, Burkholderia species, Cupriavidus species, Derxia species, Herbaspirillum species, Hydrogenophaga species, Lactobacillus species, Mesorhizobium species, Methylibium species, Methylocapsa species, Methyloferula species, Methyloversatilis species, Microcyclus species, Nitrosococcus species, Nocardia species, Oligotropha species, Pannonibacter species, Paracoccus species, Pelagibaca species, Pseudomonas species, Pseudooceanicola species, Ralstonia species, Renobacter species, Rhizobium species, Rhodobacter species, Rhodomicrobium species, Rubrivivax species, Salipiger species, Sinorhizobium species, Skermanella species, Stappia species, Thauera species, Variovorax species, Xanthobacter species, and combinations thereof.

In some aspects, the microorganism comprises or is selected from the group consisting of Acidiphilium multivorum, Alcaligenes paradoxus, Azoarcus indigens, Azohydromonas australica, Azohydromonas lata, Azorhizobium caulinodans, Azospirillium brasiliense, Azospirillum amazonsense, Azospirillum lipoferum, Azospirillum lipoferum (RSAL0111), Azospirillum thiophilum, Azotobacter chroococum (MCC 0055), Azotobacter vinelandii, Azotobacter vinelandii (RSAV006), Bacillus megaterium, Bacillus pumilus, Bacillus amyloliquefaciens, Bacillus lichenformis, Bacillus subtilis, Beggiatoa alba, Beijerinckia mobilis, Bradyrhizobium elnakii, Bradyrhizobium japonicum, Bradyrhizobium japonicum (strain USDA 122), Burkholderia vietnameiensis, Cupriavidus necator, Derxia gummosa, Gluconacetobacter diazotrophicus, Gluconacetobacter diazotrophicus (MCC 0046), Herbaspirillum autrotrophicum, Herbaspirillum frisingense (MCC 0052), Hydrogenophaga pseudoflava, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Methylibium petroleiphilum, Methylocapsa aurea, Methyloferula stellate, Methyloversatilis universalis, Microcyclus aquaticus, Microcyclus ebruneus, Nitrosococcus oceani, Nitrosomonas communis, Nitrospirillum amazonense, Nocardia autotrophica, Nocardia opaca, Oligotropha carboxidovorans, Paenibacillus durus (MCC 0046), Pannonibacter phragmitetus, Paracoccus denitrificans, Paracoccus pantrophus, Paracoccus yeei, Pelagibaca bermudensis, Pseudomonas facilis, Pseudomonas fluorescens, Pseudooceanicola atlanticus, Ralstonia eutropha, Renobacter vacuolatum, Rhizobium gallicum, Rhizobium japonicum, Rhizobium japonicum (MCC 0071), Rhizobium leguminosarum, Rhizobium leguminosarum biovar viciae, Rhodobacter capsulatus, Rhodobacter sphaeroides, Rhodomicrobium vannielii, Rubrivivax gelatinosus, Salipiger mucosus, Sinorhizobium americanum, Sinorhizobium fredii, Sinorhizobium meliloti, Skermanella stibiiresistens, Stappia aggregate, Thauera humireducens, Variovorax paradoxus, Xanthobacter autotrophicus, and combinations thereof.

In some aspects, the biofertilizer is in liquid formulation.

In some aspects, the biofertilizer is in a dry formulation.

In some aspects, the primer is in liquid formulation.

In some aspects, the primer is in a dry formulation. In some aspects, the primer is a powder.

In some aspects, the microorganism is in a powder.

In some aspects, the microorganism is lyophilized.

In some aspects, the microorganism is freeze-dried. Freeze-drying involves, for example, the removal of liquid from the microorganism.

In some aspects, the microorganism has been dried by spray drying. Spray drying generally produces small liquid droplets of specific sizes from a liquid or slurry using a spray nozzle or atomizer. After the droplets exit the nozzle or atomizer, they are dried, generally using hot air, to form a powder. Machines known as spray dryers are normally used for this process.

Primers or microorganisms (generally liquid, but also semi-solid or solid) may be dried, dehydrated or desiccated using a variety of methods. In some examples, a liquid sample may be left open so that moisture from the sample is evaporated into the air. This may be called air drying. In some examples, a gas stream (e.g., air) may apply heat to the sample by convection and moisture/vapor is removed as humidity. In some aspects, vacuum drying, where heat is supplied to the sample by conduction, radiation, or microwaves, vapor is produced and carried away by a vacuum system, may be used. In some aspects, drum drying, where a surface supplies heat to the sample, vapor is produced and carried away by an aspirator, may be used. In some aspects, a dried sample may be produced by draining (e.g., centrifugation to mechanically extract a solvent).

In some aspects, both the primer and microorganisms are in a dry formulation (e.g., both before being combined to form a composition, and within the composition).

In some aspects, both the primer and microorganisms are in a liquid formulation (e.g., both before being combined to form a composition, and within the composition).

In some aspects, the primer is in a liquid formulation and the microorganisms are in a dry formulation prior to being combined to form a composition.

In some aspects, the primer is in a dry formulation and the microorganisms are in a liquid formulation prior to being combined to form a composition.

In some aspects, the liquid formulation is concentrated to remove water.

In some aspects, the kit comprises about 50 μg to about 150 μg of primer per 1 mL of the biofertilizer.

In some aspects, the kit comprises about 50 μg to about 150 μg, about 75 μg to about 150 μg, about 100 μg to about 150 μg, about 125 μg to about 150 μg, about 50 μg to about 125 μg, about 50 μg to about 100 μg, about 50 μg to about 75 μg, about 75 μg to about 125 μg, about 75 μg to about 100 μg, or about 100 μg to about 125 μg of primer per 1 mL of the biofertilizer.

In some aspects, the kit comprises about 50 μg, about 75 μg, about 100 μg, about 125 μg, or about 150 μg of primer per 1 mL of the biofertilizer.

In some aspects, the kit comprises about 100 μg of primer per 1 mL of the biofertilizer.

In some aspects, the kit comprises about 0.05 g to about 0.15 g of primer per 1 L of the biofertilizer.

In some aspects, the kit comprises about 0.05 g to about 0.15 g, about 0.075 g to about 0.15 g, about 0.1 g to about 0.15 g, about 0.125 g to about 0.15 g, about 0.05 g to about 0.125 g, about 0.05 g to about 0.1 g, about 0.05 g to about 0.075 g, about 0.075 g to about 0.125 g, about 0.1 g to about 0.125 g, about 0.075 g to about 0.1 g, about 0.05 g to about 0.5 g, about 0.05 g to about 1 g, about 0.05 g to about 1.5 g, about 0.05 g to about 2 g, about 0.05 g to about 2.5 g, about 0.05 g to about 3 g, about 0.05 g to about 4 g, about 0.05 g to about 5 g, about 0.05 g to about 6 g, about 0.05 g to about 7 g, about 0.05 g to about 8 g, about 0.05 g to about 9 g, or about 0.05 g to about 10 g of primer per 1 L of the biofertilizer.

In some aspects, the kit comprises about 0.05 g, about 0.075 g, about 0.1 g, about 0.125 g, about 0.15 g, about 0.25 g, about 0.5 g, about 1 g, about 1.5 g, about 2 g, about 2.5 g, about 3 g, about 4 g, about 5 g, about 6 g, about 7 g, about 8 g, about 9 g, or about 10 g of primer per 1 L of the biofertilizer.

In some aspects, the kit comprises about 0.1 g of primer per 1 L of the biofertilizer.

In some aspects, the kit comprises about 5% w/v of the primer.

In some aspects, the kit comprises about 1% w/v to about 6% w/v, about 1% w/v to about 5% w/v, about 1% w/v to about 4% w/v, about 1% w/v to about 3% w/v, about 1% w/v to about 2% w/v, about 2% w/v to about 6% w/v, about 3% w/v to about 6% w/v, about 4% w/v to about 6% w/v, about 2% w/v to about 5% w/v, about 2% w/v to about 4% w/v, about 2% w/v to about 3% w/v, about 3% w/v to about 5% w/v, or about 3% w/v to about 4% w/v of the primer.

In some aspects, the kit comprises about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, or about 6% w/v of the primer.

In some aspects, the kit comprises about 2.5% w/v of the primer.

In some aspects, the kit comprises about 21.5 μg/mL of the primer.

In some aspects, the amino acid is arginine.

In some aspects, the kit comprises about 15 μg/mL to about 25 μg/mL, 15 μg/mL to about 20 μg/mL, 20 μg/mL to about 25 μg/mL, 21.5 μg/mL to about 25 μg/mL, 15 μg/mL to about 21.5 μg/mL, 17.5 μg/mL to about 21.5 μg/mL, or 21.5 μg/mL to about 23 μg/mL of the primer.

In some aspects, the kit comprises about 15 μg/mL, 17.5 μg/mL, 21.5 μg/mL, about 23 μg/mL, or about 25 μg/mL of the primer.

In some aspects, the kit comprises about 2% w/v to about 3% w/v of the primer, optionally about 2.8% w/v of the primer.

In some aspects, the kit comprises about 2.8% w/v of the primer.

In some aspects, the amino acid is asparagine.

In some aspects, the kit comprises about 0.5% w/v to about 1% w/v of the primer, optionally about 0.6% w/v of the primer.

In some aspects, the kit comprises about 0.5% w/v to about 2% w/v, about 1% w/v to about 2% w/v, about 1.5% w/v to about 2% w/v, about 0.5% w/v to about 1.5% w/v, or about 0.5% w/v to about 1% w/v of the primer.

In some aspects, the kit comprises about 0.5% w/v, about 1% w/v, about 1.5% w/v, or about 2% w/v of the primer.

In some aspects, the kit comprises about 0.6% w/v of the primer.

In some aspects, the amino acid is glutamic acid.

In some aspects, the kit comprises about 10% w/v to about 20% w/v of the primer, optionally about 14.6% w/v of the primer.

In some aspects, the kit comprises about 10% w/v to about 25% w/v, about 10% w/v to about 22.5% w/v, about 10% w/v to about 20% w/v, about 10% w/v to about 17.5% w/v, about 10% w/v to about 15% w/v, about 10% w/v to about 12.5% w/v, about 12.5% w/v to about 20% w/v, about 15% w/v to about 20% w/v, about 17.5% w/v to about 20% w/v, about 17.5% w/v to about 25% w/v, or about 20% w/v to about 25% w/v of the primer.

In some aspects, the kit comprises about 10% w/v, about 12.5% w/v, about 15% w/v, about 17.5% w/v, about 20% w/v, or about 25% w/v of the primer.

In some aspects, the kit comprises about 14.6% w/v of the primer.

In some aspects, the amino acid is glycine.

In some aspects, the kit comprises about 1.5% w/v to about 3% w/v of the primer, optionally about 2.3% w/v of the primer.

In some aspects, the kit comprises about 2.3% w/v of the primer.

In some aspects, the amino acid is tryptophan.

In some aspects, the kit comprises about 0.5% w/v to about 1% w/v of the primer, optionally about 0.7% w/v of the primer.

In some aspects, the kit comprises about 0.7% w/v of the primer.

In some aspects, the amino acid is a combination of the amino acids.

In some aspects, the kit comprises about 15% w/v to about 25% w/v of the primer, optionally about 21% w/v of the primer.

In some aspects, the kit comprises about 21% w/v of the primer.

In some aspects, the kit comprises about 25 mg/mL to about 75 mg/mL of the primer, optionally about 50 mg/mL of the primer.

In some aspects, the kit comprises about 50 mg/mL of the primer.

In some aspects, the kit comprises about 25 mg/mL to about 75 mg/mL, about 25 mg/mL to about 65 mg/mL, about 25 mg/mL to about 55 mg/mL, about 25 mg/mL to about 50 mg/mL, about 25 mg/mL to about 45 mg/mL, about 25 mg/mL to about 35 mg/mL, about 35 mg/mL to about 75 mg/mL, about 45 mg/mL to about 75 mg/mL, about 50 mg/mL to about 75 mg/mL, about 55 mg/mL to about 75 mg/mL, about 65 mg/mL to about 75 mg/mL, about 35 mg/mL to about 50 mg/mL, or about 50 mg/mL to about 65 mg/mL of the primer.

In some aspects, the kit comprises about 25 mg/mL, about 35 mg/mL, about 45 mg/mL, about 50 mg/mL, about 55 mg/mL, about 65 mg/mL, or about 75 mg/mL of the primer.

In some aspects, the kit comprises about 0.025 M to about 0.05 M of the primer, optionally about 0.03 M of the primer.

In some aspects, the kit comprises about 0.5 M to about 1.5 M of the primer, optionally about 1.0 M of the primer.

In some aspects, the kit comprises about 0.5 M to about 1.5 M, about 1.0 M to about 1.5 M, or about 0.5 M to about 1.0 M of the primer.

In some aspects, the kit comprises about 0.5 M, about 1.0 M, or about 1.5 M of the primer.

In some aspects, the kit comprises about 0.03 M of the primer.

In some aspects, the kit comprises about 1.0 M of the primer.

In some aspects, the primer is thiamine.

In some aspects, the kit comprises about 3 μg/mL to about 4 μg/mL of the primer, optionally about 3.3 μg/mL of the primer.

In some aspects, the kit comprises about 2 μg/mL to about 6 μg/mL, about 3 g/mL to about 6 μg/mL, about 4 μg/mL to about 6 μg/mL, about 5 μg/mL to about 6 g/mL, about 2 μg/mL to about 5 μg/mL, about 2 μg/mL to about 4 μg/mL, about 2 g/mL to about 3 μg/mL, about 3 μg/mL to about 4 μg/mL, about 3.5 μg/mL to about 4 g/mL, or about 3 μg/mL to about 3.5 μg/mL of the primer.

In some aspects, the kit comprises about 2 μg/mL, about 3 μg/mL, about 3.5 g/mL, about 4 μg/mL, about 5 μg/mL, or about 6 μg/mL of the primer.

In some aspects, the kit about 3.3 μg/mL of the primer.

In some aspects, the kit comprises about 6 μg/mL to about 8 μg/mL of the primer, optionally about 6.7 μg/mL of the primer.

In some aspects, the kit about 6.7 μg/mL of the primer.

In some aspects, the kit comprises about 6 μg/mL to about 8 μg/mL, about 6 g/mL to about 7 μg/mL, or about 7 μg/mL to about 8 μg/mL of the primer.

In some aspects, the kit comprises about 6 μg/mL, about 7 μg/mL, or about 8 g/mL of the primer.

In some aspects, the primer is pantothenic acid.

In some aspects, the kit comprises about 2 μg/mL to about 3 μg/mL of the primer, optionally about 2.2 μg/mL of the primer.

In some aspects, the kit comprises about 3 μg/mL to about 5 μg/mL of the primer, optionally about 4.3 μg/mL of the primer.

In some aspects, the kit comprises about 20 μg/mL to about 25 μg/mL of the primer, optionally about 21.5 μg/mL of the primer.

In some aspects, the kit comprises about 20 μg/mL to about 25 μg/mL, about 15 μg/mL to about 30 μg/mL, about 15 μg/mL to about 25 μg/mL, about 15 μg/mL to about g/mL, about 20 μg/mL to about 30 μg/mL, about 25 μg/mL to about 30 μg/mL or about 15 μg/mL to about 25 μg/mL of the primer.

In some aspects, the kit comprises about 15 μg/mL, about 20 μg/mL, about 25 g/mL, or about 30 μg/mL of the primer.

In some aspects, the kit comprises about 2.2 μg/mL of the primer.

In some aspects, the kit comprises about 4.3 μg/mL of the primer.

In some aspects, the kit comprises about 21.5 μg/mL of the primer.

In some aspects, the primer is a combination of thiamine and pantothenic acid.

In some aspects, the kit comprises about 0.7 μg/mL of thiamine and about 4.3 g/mL of pantothenic acid.

In some aspects, the kit comprises about 0.5 μg/mL to about 1.0 μg/mL, about 0.75 μg/mL to about 1.0 μg/mL, or about 0.5 μg/mL to about 0.75 μg/mL of thiamine.

In some aspects, the kit comprises about 0.5 μg/mL, about 0.75 μg/mL, or about 1.0 μg/mL of thiamine.

In some aspects, the kit comprises about 4 μg/mL to about 6 μg/mL, about 5 g/mL to about 6 μg/mL, or about 4 μg/mL to about 5 μg/mL of pantothenic acid.

In some aspects, the kit comprises about 4 μg/mL, about 5 μg/mL, or about 6 g/mL of pantothenic acid.

In some aspects, the kit comprises about 0.010 mM to about 0.1 mM of the primer, optionally about 0.016 mM, about 0.064 mM, or about 0.080 mM of the primer.

In some aspects, the kit comprises about 0.010 mM to about 0.1 mM, about 0.010 mM to about 0.070 mM, about 0.010 mM to about 0.050 mM, about 0.010 mM to about 0.030 mM, about 0.030 mM to about 0.1 mM, about 0.050 mM to about 0.1 mM, or about 0.070 mM to about 0.1 mM of the primer.

In some aspects, the kit comprises about 0.010 mM, about 0.030 mM, about 0.050 mM, about 0.070 mM, or about 0.1 mM of the primer.

In some aspects, the kit comprises about 1.0 g/L or about 4.0 g/L of the primer.

In some aspects, the composition comprises about 1.0 g/L of the primer.

In some aspects, the kit comprises about 4.0 g/L of the primer.

In some aspects, the kit further comprises instructions for applying the primer and biofertilizer to a plant.

In some aspects, the plant is a lettuce (e.g., Lactuca sativa), a tomato (e.g., Solanum lycopersicum), a clover (e.g., Trifolium repens), a broccoli (e.g., Brassica oleracea), a corn (e.g., Zea mays), or a creeping bluegrass (e.g., Poa reptans).

In some aspects, the plant is an Asteraceae, a Brassicaceae, a Fabaceae, a Poaceae, or a Solanaceae.

### Methods of Use

Certain aspects of the disclosure provide a method of increasing nitrogen utilization efficiency of a plant, comprising administering to the plant any of the compositions, diluents, or microorganisms disclosed herein.

Certain aspects of the disclosure provide a method of priming nitrogen fixation, comprising applying a primer to a biofertilizer comprising a microorganism, wherein the primer comprises glucose, fructose, or a combination thereof, and wherein the microorganism is Xanthobacter autotrophicus.

Certain aspects of the disclosure provide a method of enhancing exopolysaccharides (EPS) production of a biofertilizer, comprising applying a primer to a biofertilizer comprising a microorganism, wherein the primer comprises glucose, fructose, or a combination thereof, and wherein the microorganism is Xanthobacter autotrophicus.

Certain aspects of the disclosure provide a method of enhancing microbial root colonization of a plant, comprising i) applying a primer to a biofertilizer comprising a microorganism, and ii) applying the primer and the biofertilizer to a plant, wherein the primer comprises glucose, fructose, or a combination thereof, and wherein the microorganism is Xanthobacter autotrophicus.

Certain aspects of the disclosure provide a method of increasing viability of a biofertilizer, comprising applying a primer to a biofertilizer comprising a microorganism, wherein the primer comprises glucose, fructose, or a combination thereof, and wherein the microorganism is Xanthobacter autotrophicus.

Certain aspects of the disclosure provide a method of priming nitrogen fixation, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of enhancing exopolysaccharides (EPS) production of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of enhancing microbial root colonization, comprising i) applying a primer to a biofertilizer comprising a microorganism, and ii) applying the primer and the biofertilizer to a plant, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of increasing viability of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

In some aspects, the primer comprises glucose.

In some aspects, the primer comprises fructose.

Certain aspects of the disclosure provide a method of priming nitrogen fixation, comprising applying a primer to a biofertilizer comprising a microorganism, wherein the microorganism is Xanthobacter autotrophicus.

Certain aspects of the disclosure provide a method of enhancing exopolysaccharides (EPS) production of a biofertilizer, comprising applying a primer to a biofertilizer comprising a microorganism, wherein the microorganism is Xanthobacter autotrophicus.

Certain aspects of the disclosure provide a method of enhancing microbial root colonization, comprising i) applying a primer to a biofertilizer comprising a microorganism, and ii) applying the primer and the biofertilizer to a plant, wherein the microorganism is Xanthobacter autotrophicus.

Certain aspects of the disclosure provide a method of increasing viability of a biofertilizer, comprising applying a primer to a biofertilizer comprising a microorganism, wherein the microorganism is Xanthobacter autotrophicus.

In some aspects, the primer is glucose.

In some aspects, the primer is fructose.

In some aspects, the sugar is sucrose.

In some aspects, the carbohydrate is molasses.

In some aspects, the primer comprises an amino acid.

In some aspects, the amino acid is glycine.

In some aspects, the amino acid is glycine, glutamic acid, arginine, asparagine, tryptophan, or any combination thereof.

In some aspects, the amino acid is glutamic acid, glycine, or tryptophan.

In some aspects, the amino acid is glutamic acid, histidine, glycine, arginine, or any combination thereof.

In some aspects, the primer comprises one or more iron-rich soluble compounds.

In some aspects, the primer comprises one or more molybdenum rich-soluble compounds.

In some aspects, the molybdenum rich-soluble compound is sodium molybdate.

In some aspects, the primer comprises an organic acid.

In some aspects, the organic acid is malic acid.

In some aspects, the primer is a salt solution.

In some aspects, the salt solution is potassium chloride.

In some aspects, the primer is pantothenic acid, thiamine, or a combination thereof.

In some aspects, the primer comprises a source of glycine.

In some aspects, the primer comprises a source of one or more amino acids.

In some aspects, the primer comprises a source of one or more iron-rich soluble compounds.

In some aspects, the primer comprises a source of one or more molybdenum rich-soluble compounds.

In some aspects, the primer is glucose.

In some aspects, the primer is fructose.

Certain aspects of the disclosure provide a method of priming nitrogen fixation, comprising applying a primer to a biofertilizer comprising a microorganism, wherein the primer comprises glucose, fructose, or a combination thereof.

Certain aspects of the disclosure provide a method of enhancing exopolysaccharides (EPS) production of a biofertilizer, comprising applying a primer to a biofertilizer comprising a microorganism, wherein the primer comprises glucose, fructose, or a combination thereof.

Certain aspects of the disclosure provide a method of enhancing microbial root colonization, comprising i) applying a primer to a biofertilizer comprising a microorganism, and ii) applying the primer and the biofertilizer to a plant, wherein the primer comprises glucose, fructose, or a combination thereof.

Certain aspects of the disclosure provide a method of increasing viability of a biofertilizer, comprising applying a primer to a biofertilizer comprising a microorganism, wherein the primer comprises glucose, fructose, or a combination thereof.

Certain aspects of the disclosure provide a method of priming nitrogen fixation, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of enhancing exopolysaccharides (EPS) production of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of enhancing microbial root colonization, comprising i) applying a primer to a biofertilizer comprising a microorganism, and ii) applying the primer and the biofertilizer to a plant, wherein the primer comprises glucose, fructose, sucrose, malic acid, pantothenic acid, thiamine, an amino acid, potassium chloride, sodium molybdate, molasses, or a combination thereof.

Certain aspects of the disclosure provide a method of increasing viability of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

In some aspects, the primer comprises glucose.

In some aspects, the primer comprises fructose.

In some aspects, the microorganism comprises or is selected from the group consisting of Acidiphilium species, Alcaligenes species, Arthrobacter species, Azohydromonas species, Azospirillum species, Azotobacter species, Bacillus species, Beggiatoa species, Beijerinckia species, Bradyrhizobium species, Burkholderia species, Cupriavidus species, Derxia species, Herbaspirillum species, Hydrogenophaga species, Lactobacillus species, Mesorhizobium species, Methylibium species, Methylocapsa species, Methyloferula species, Methyloversatilis species, Microcyclus species, Nitrosococcus species, Nocardia species, Oligotropha species, Pannonibacter species, Paracoccus species, Pelagibaca species, Pseudomonas species, Pseudooceanicola species, Ralstonia species, Renobacter species, Rhizobium species, Rhodobacter species, Rhodomicrobium species, Rubrivivax species, Salipiger species, Sinorhizobium species, Skermanella species, Stappia species, Thauera species, Variovorax species, Xanthobacter species, and combinations thereof.

In some aspects, the microorganism comprises or is selected from the group consisting of Acidiphilium multivorum, Alcaligenes paradoxus, Azoarcus indigens, Azohydromonas australica, Azohydromonas lata, Azorhizobium caulinodans, Azospirillium brasiliense, Azospirillum amazonsense, Azospirillum lipoferum, Azospirillum lipoferum (RSAL0111), Azospirillum thiophilum, Azotobacter chroococum (MCC 0055), Azotobacter vinelandii, Azotobacter vinelandii (RSAV006), Bacillus megaterium, Bacillus pumilus, Bacillus amyloliquefaciens, Bacillus lichenformis, Bacillus subtilis, Beggiatoa alba, Beijerinckia mobilis, Bradyrhizobium elnakii, Bradyrhizobium japonicum, Bradyrhizobium japonicum (strain USDA 122), Burkholderia vietnameiensis, Cupriavidus necator, Derxia gummosa, Gluconacetobacter diazotrophicus, Gluconacetobacter diazotrophicus (MCC 0046), Herbaspirillum autrotrophicum, Herbaspirillum frisingense (MCC 0052), Hydrogenophaga pseudoflava, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Methylibium petroleiphilum, Methylocapsa aurea, Methyloferula stellate, Methyloversatilis universalis, Microcyclus aquaticus, Microcyclus ebruneus, Nitrosococcus oceani, Nitrosomonas communis, Nitrospirillum amazonense, Nocardia autotrophica, Nocardia opaca, Oligotropha carboxidovorans, Paenibacillus durus (MCC 0046), Pannonibacter phragmitetus, Paracoccus denitrificans, Paracoccus pantrophus, Paracoccus yeei, Pelagibaca bermudensis, Pseudomonas facilis, Pseudomonas fluorescens, Pseudooceanicola atlanticus, Ralstonia eutropha, Renobacter vacuolatum, Rhizobium gallicum, Rhizobium japonicum, Rhizobium japonicum (MCC 0071), Rhizobium leguminosarum, Rhizobium leguminosarum biovar viciae, Rhodobacter capsulatus, Rhodobacter sphaeroides, Rhodomicrobium vannielii, Rubrivivax gelatinosus, Salipiger mucosus, Sinorhizobium americanum, Sinorhizobium fredii, Sinorhizobium meliloti, Skermanella stibiiresistens, Stappia aggregate, Thauera humireducens, Variovorax paradoxus, Xanthobacter autotrophicus, and combinations thereof.

In some aspects, the microorganism is Xanthobacter autotrophicus (e.g., Xanthobacter autotrophicus 7c).

In some aspects, the primer is added to the biofertilizer prior to applying the biofertilizer to a plant.

In some aspects, the primer is added to the biofertilizer at the same time as applying the biofertilizer to a plant.

In some aspects, the plant is a lettuce (e.g., Lactuca sativa), a tomato (e.g., Solanum lycopersicum), a clover (e.g., Trifolium repens), a broccoli (e.g., Brassica oleracea), or a creeping bluegrass (e.g., Poa reptans).

In some aspects, the plant is Lactuca sativa var. salivus.

In some aspects, the plant is an Asteraceae, a Brassicaceae, a Fabaceae, a Poaceae, or a Solanaceae.

### EXAMPLES

### Example 1. Nitrogen Fixation In Vitro

The purpose of this experiment was to observe the ability of X. autotrophicus to fix nitrogen in vitro, both with and without the addition of carbon from sources commonly found in the rhizosphere. Such sources include sugars such as fructose and glucose, and organic acids such as citric acid, malic acid, and oxalic acid.

The objective of this experiment was to quantify and compare the nitrogen-fixing ability of X. autotrophicus in vitro, with and without the addition of fructose, glucose, citric acid, malic acid, or oxalic acid.

### Experimental Design

Xanthobacter autotrophicus 7c (NCMA B104) was used for all experiments described below. X. autotrophicus was grown either in flasks or bioreactor, as specified below. Glycerol stocks of X. autotrophicus were streaked onto nutrient broth (NB) agar (Difco™, VWR, West Chester, PA). Flask cultures of liquid NB, prepared according to manufacturer instructions, were then inoculated with isolated colonies selected from NB agar plates. Flasks were placed on an orbital shaker at 200 rpm and cultures were grown at 30° C. before use in the experiments described below. Bioreactor batches were grown as previously described by Liu, C., et al., PNAS 114:6450-6455 (2017).

¹⁵N₂ culture incubations were performed as described in Smercina, D. N., et al., Plant and Soil 445:595-611 (2019) with adaptations for liquid culture incubations. Briefly, 20 mL glass vials equipped with PTFE/silicon septa (Supelco, St. Louis, MO) were filled with 9.5 mL of X. autotrophicus bioreactor culture plus 0.5 ml of sterile water or carbon solution according to the assigned treatment. Carbon (C) solutions of fructose, glucose, citric acid, malic acid, or oxalic acid were prepared at 2 mg C mL⁻¹ such that added C was equivalent to 100 μg C mL⁻¹ in the incubation vials. This addition rate was chosen to represent measured rates of C exudation from roots (Baudoin, E., et al., Soil Biology and Biochemistry 35:1183-1192 (2003)).

Replicate vials of each treatment were prepared for natural abundance reference vials and ¹⁵N-enriched incubation. For enriched incubation, 6 mL of headspace was removed using a syringe and replaced with 98 atom % ¹⁵N₂ (Sigma-Aldrich, St. Louis, MO) generating an incubation atmosphere of 49 atom % ¹⁵N. All vials were incubated for 3 days at 30° C. on an orbital shaker at 100 rpm. At harvest, vial volumes were transferred to 15 ml tubes. Tubes were centrifuged at 2500 g for 10 minutes to pellet cells, supernatant was removed and discarded, and pellets were dried at 60° C. for 48 hours until completely dry. Samples were prepared and analyzed via Isotope Ratio Mass Spectrometry (IRMS) following standard procedures at University of Arkansas Stable Isotope Laboratory (Fayetteville, AR).

### Results

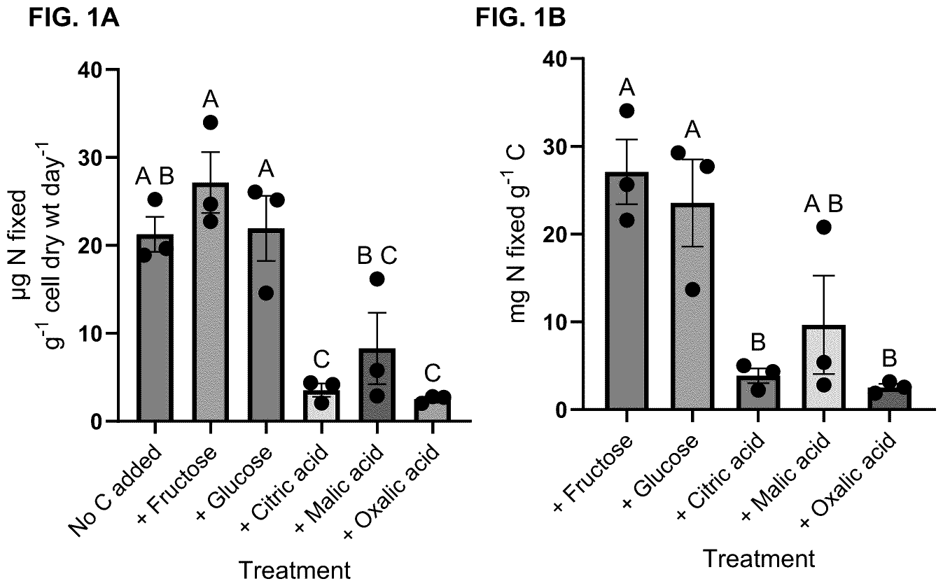

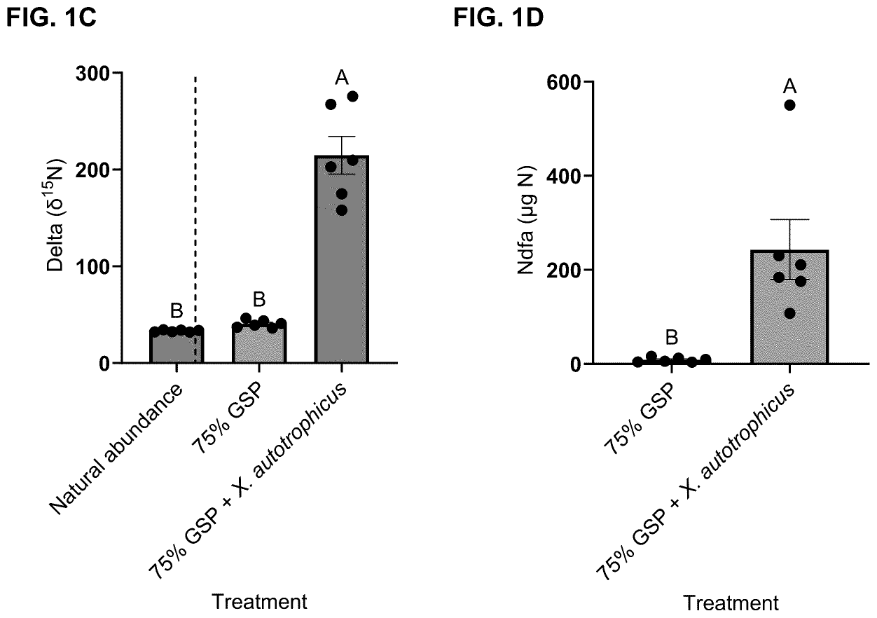

The purpose of this experiment was to observe the ability of X. autotrophicus to fix nitrogen in vitro, with and without the addition of fructose, glucose, citric acid, malic acid, or oxalic acid. When assessed in controlled liquid conditions, X. autotrophicus was found to fix an average of 21.2 μg N g⁻¹ cell dry weight day⁻¹ into cellular biomass when no additional carbon was provided (FIG. 1A). When additional carbon was provided, the rate of nitrogen fixation differed from baseline (i.e., no carbon added), but the direction depended on the carbon source. The sugars, fructose and glucose, were found to slightly increase nitrogen fixation rates, while the organic acids, citric acid, malic acid, and oxalic acid, tended to decrease nitrogen fixation rates on a per cell dry weight basis (FIG. 1A).

The nitrogen fixation rates of the treatments with carbon were normalized to a per gram of carbon basis (FIG. 1B). When sugars were provided, X. autotrophicus fixed an average of 23.5-27.1 mg N g⁻¹ C added, which is over 3× greater than reported for Azotobacter vinelandii, where A. vinelandii was fixed 7 mg N g⁻¹ C when provided sucrose as a carbon source (Hill, S., Biological Nitrogen Fixation 87-134 (1992)). When organic acids were provided, X. autotrophicus fixed an average of 2.5-9.7 mg N g⁻¹ C. As a comparison, X. autotrophicus supplied with malic acid as a C source fixed 9.7 mg N g⁻¹ C on average, while Azospirillum brasilense given the same carbon source fixed 26 mg N g⁻¹ C (Hill, 1992). These findings demonstrate the ability of X. autotrophicus to fix nitrogen at rates similar to, and often greater than, many other well-known non-symbiotic diazotrophs.

### Example 2. Nitrogen Fixation and Transfer in Planta

The objective of this experiment was to quantify the transfer of fixed nitrogen from X. autotrophicus to lettuce plants by adapting the ¹⁵N₂ incorporation method for whole plant incubations.

### Experimental Design

Romaine lettuce seedlings (Lactuca sativa var. salivus, Johnny's Selected Seeds, Winslow, ME) were sown into germination trays filled with Sunshine® Mix #1 (Sungro Horticulture, Agawam, MA). Trays were placed in a growth chamber with 16-hour day/8-hour night light cycles, temperatures at 25° C. during the day and 22° C. at night, and humidity maintained at 50%. Seedlings were grown for 21 days in trays before transplanting into glass jars. Glass jars (32 oz, Ball Corp., Broomfield, CO, USA) were filled with a single layer of glass marbles topped with 100 g of moist coconut coir (CANNA Coco Brick, CANNA, Arcadia, CA).

After transplant, lettuce plants were returned to the growth chamber for an additional 21 days, including a 7-day ¹⁵N₂ incorporation incubation. During the first 14 days, jars remained unsealed and open to the air. Plants received weekly applications of nitrogen-free Hoagland's solution for micronutrients, nitrogen fertilizer as calcium nitrate (YaraLiva, Tampa, FL) equivalent to 75% of grower standard practice (GSP), and either X. autotrophicus (treated plants) or sterile water (untreated plants). 75% GSP for the growth period of this experiment was equivalent to 36 lbs. N ac-1 and created conditions through which plants could rely on nitrogen fixation by X. autotrophicus as a supplemental nitrogen source.

X. autotrophicus was directly applied at 3.33e9 CFU per application for a total of 1e10 CFU per plant. Following the last application of X. autotrophicus, jars were sealed with standard lids equipped with butyl rubber septa. Immediately after sealing, 50 mL of headspace was removed via syringe and replaced with 98 atom % ¹⁵N₂, creating an atmosphere enriched to 14.4 atom % ¹⁵N. In addition to enriched jars that received ¹⁵N₂, an additional set of 75% GSP untreated jars were incubated under natural abundance conditions.

Sealed jars were incubated in the growth chamber for 7 days during which CO₂ levels were monitored daily using a Licor LI-7000 CO₂/H₂O analyzer and replenished via syringe using UHP CO₂ as needed. After 7 days, the aboveground leaf material was harvested. Samples were dried at 60° C. for 48 hours until completely dry. Samples were prepared and analyzed via Isotope Ratio Mass Spectrometry (IRMS) following standard procedures at the Marine Biological Laboratory Stable Isotope Laboratory (Woods Hole, MA).

### Results

Lettuce was grown in sterile, quart (0.94 L) glass jars equipped with airtight lids outfitted with septa for gas additions. This system allowed lettuce to be grown for 3 weeks including a 1-week incubation in which jars were sealed and the headspace was enriched with ¹⁵N₂. All plants received standard additions of micronutrients, as N-free Hoagland's solution, but reduced N at only 75% of grower standard practice (GSP). This 25% reduction in available N created conditions through which the plant could rely on N-fixation by X. autotrophicus as a supplemental N source. A subset of plants was then treated with a total of 1e10 CFU of X. autotrophicus per plant. Using this approach, ¹⁵N fixed by X. autotrophicus was followed from the atmosphere into lettuce leaf tissue, demonstrating in planta exchange of fixed N. The purpose of this experiment was to quantify the amount of fixed nitrogen transferred from X. autotrophicus to lettuce plants. There was a significant increase in the delta ¹⁵N signature (δ¹⁵N) of leaf tissue when X. autotrophicus was present compared to when it was not (FIG. 1C), indicating that nitrogen fixation directly contributed to the uptake of nitrogen enriched with ¹⁵N in the lettuce plants.

The δ¹⁵N values and leaf tissue nitrogen content were used to calculate the nitrogen derived from atmosphere (Ndfa) in the leaf tissue (Warembourg, F. R., et al., Nitrogen Isotope Techniques 654:157-180 (1993); Weaver, R. W., and Danso, S. K. A., Microbiological and Biochemical Properties 5:1019-1045 (1994)). On average, the Ndfa in the experimental system was 243.0 μg N over 7 days (FIG. 1D). Assuming this rate was consistent over the entire growth period, the uptake of nitrogen fixed by X. autotrophicus met up to 12% of the plant nitrogen deficit and provided on average ˜1.5% of total nitrogen in leaf tissue. This is the first demonstration of direct transfer of recently fixed N from X. autotrophicus to a nearby plant and is a critical validation of this microbe's potential as a biofertilizer.

### Example 3. Root Development Assay

The objective of this experiment was to characterize the root development of lettuce seedlings treated with X. autotrophicus compared to treatment with water.

### Experimental Design

Root development was assessed in romaine lettuce seedlings (Lactuca sativa var. salivus, Johnny's Selected Seeds, Winslow, ME) using an adaptation of previously described plate methods (Zamioudis, C., et al., Plant Physiology 162:304-318 (2013); Wintermans, P. C. A., et al., Plant Molecular Biology, (2016)). Briefly, 50% Hoagland's media prepared with 1.5% agar was adjusted to pH 6.5 and poured into 100 mm×100 mm square plates. Lettuce seeds were prepared for plating by first surface sterilizing and stratifying as follows: a 2 ml sterile microcentrifuge tube was filled with 500 μl of seeds along with 1 ml of 50% bleach (Lindsey III, B. E., et al., Journal of Visualized Experiments e56587 (2017)). Seeds were exposed to bleach solution for 10 minutes, after which the bleach solution was aspirated, and the seeds were washed with 1 ml of sterile water six times. Seeds were then resuspended 1 ml sterile water and placed at 4° C. for 48 hours to stratify, after which the water was aspirated, and seeds were stored dry at 4° C. for up to 2 weeks before sowing.

To confirm sterilization, at least 3 lettuce seeds were placed aseptically onto NB agar and monitored for microbial growth for up to 1 week. Sterile lettuce seeds were placed near the top of the 50% Hoagland's agar plate along the “sowing line”. Using a pipette, 30 μl of either water or a 1.3e9 CFU ml-1 X. autotrophicus culture, was plated in a single line 45 mm below the sowing line. Plates were then placed in a growth chamber with 16-hour day/8-hour night light cycles, temperatures at 25° C. during the day and 22° C. at night, and humidity maintained at 50%. Plates were kept at an ˜80° angle during growth to encourage root growth along the agar surface.

Seedlings were grown for 7 days and then root development was assessed, including tap root length (measured in cm) and counts of lateral root number. Lateral roots emerging from the taproot were identified as secondary roots arisen from the taproot's pericycle, as compared to root hairs that are subcellular projections. Lateral root density was also determined by dividing the lateral root number of total tap root length.

### Results

Lettuce seedlings exposed to X. autotrophicus showed significant decreases in tap root length and a statistically significant increase in lateral root density (number of roots cm⁻¹ tap root). These responses are characteristic of root exposure to increased ammonium. When coupled with the confirmed ability of X. autotrophicus to fix nitrogen both in vitro and in planta, as described above, these observed impacts on root development serve to further support that X. autotrophicus is fixing atmospheric nitrogen in the rhizosphere and releasing ammonium for plant uptake (Data not shown).

### Example 4. Root Colonization on Lettuce

The purpose of this experiment was to observe the ability of X. autotrophicus to colonize lettuce roots.

### Experimental Design

For microscopy purposes, a green fluorescent protein (GFP)-expressing X. autotrophicus strain was generated as follows. The prpsM-GFP plasmid (6402 bp) was constructed by cloning the GFP gene into the multi-cloning site of the pCM66T plasmid, using the constitutive rpsM promoter from X. autotrophicus GJ10, to drive expression. The P1RFP plasmid was obtained from the Rebecca Sherbo laboratory (Sherbo, R. S., et al., PNAS 119:e2210538119 (2022)), and the EGFP-pBAD (Plasmid #54762) was purchased from Addgene (Watertown, MA).

DNA was PCR-amplified with Phusion High-Fidelity DNA polymerase (ThermoFisher Scientific, Waltham, MA) followed by agarose gel electrophoresis. Correct DNA bands were extracted using the Monarch DNA gel extraction kit (New England Biolabs, Ipswich, MA) and assembled via Gibson assembly (Invitrogen, Waltham, MA). Specifically, primers 2303 and 2304 were used to amplify GFP, 2305 and 2306 were used to clone the plasmid backbone.

E. coli strain DH5a (ThermoFisher Scientific, Waltham, MA) was transformed using a standard heat shock protocol. Transformants were selected on Luria broth (LB) agar plates supplemented with 40 μg mL-1 kanamycin and further screened for the presence of an inserted GFP fragment by blue-white screening. Plasmid constructs were confirmed by Sanger sequencing using primers 2301 and 2302. Plasmids were purified using a plasmid DNA miniprep kit (ThermoFisher Scientific, Waltham, MA) following the manufacturer's instructions.

Plasmids were introduced into X. autotrophicus by electroporation, following the method previously described by Swaving, J., et al., Journal of Microbiological Methods 25:343-348 (1996). Following electroporation, X. autotrophicus cultures were plated on NB agar plates supplemented with 20 μg mL-1 kanamycin and allowed to grow for three days at 30° C. Fluorescent protein expression was visualized using the EVOS M7000 imaging system (ThermoFisher Scientific, Waltham, MA) under the GFP channel (488 nm).

GFP X. autotrophicus were first streaked onto NB agar supplemented with 40 μg mL-1 kanamycin and then isolated colonies were transferred to NB liquid media supplement with 40 μg mL-1 kanamycin. Before application to plants, liquid cultures of GFP X. autotrophicus were pelleted and washed of growth media to remove any residual kanamycin before resuspending in phosphate buffered saline (PBS).

Surface sterilized and pre-germinated romaine lettuce seedlings were grown in GA-7 Magenta™ vessels (Merck KGaA, Darmstadt, Germany) with 50% Hoagland's agar (0.6%) supplemented with 1 g C L-1 glucose for 5-weeks. Seeds were surface sterilized as described above and then plated on 1.0% agar to germinate. Individual pre-germinated seedlings were then aseptically transferred to Magenta™ vessels and placed on the agar surface in the center of the vessel. Two treatments were investigated: (1) untreated control, in which seedlings were spotted with 10 μl of sterile water and (2) X. autotrophicus-inoculated, in which seedlings were spotted with 10 μl of culture containing 1e7 CFU of GFP X. autotrophicus. After applying treatments, Magenta™ vessels were sealed and placed in a growth chamber with 16-hour day/8-hour night light cycles, temperatures at 25° C. during the day and 22° C. at night, and humidity maintained at 50%. Plant growth was visually monitored over the course of 5-weeks after which roots inside the agar were imaged using a Leica M205 FCA Stereomicroscope equipped with fluorescence channels (Leica, Deerfield, IL). Roots were imaged under brightfield and GFP fluorescence (488 nm).

### Results

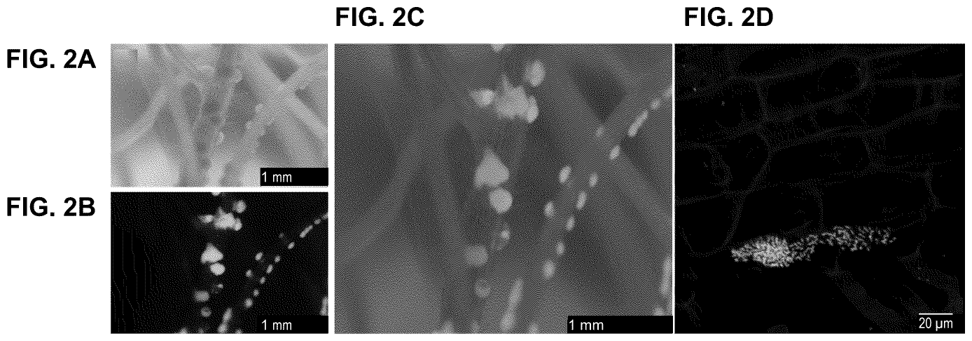

The purpose of this experiment was to observe the colonization of X. autotrophicus on inoculated lettuce roots. Colonies of X. autotrophicus were found throughout the root system/rhizosphere of lettuce (FIGS. 2A-2C), indicating that X. autotrophicus had successfully colonized and grown in the lettuce rhizosphere.

Additionally, using confocal fluorescence microscopy of lettuce seedling roots, X. autotrophicus colonies were observed interacting closely with lettuce roots and colonizing around root hairs (FIG. 2D). Root hairs represent a critical habitat commonly colonized by plant beneficial microorganisms.

Together with the findings from the experiments discussed above, these observations of lettuce root colonization suggest X. autotrophicus delivers nitrogen through close interactions with plant roots.

### Example 5. Root Colonization on Additional Plants

The purpose of this experiment was to observe the ability of X. autotrophicus to colonize the roots of additional plant species, including tomato (Solanum lycopersicum), clover (Trifolium repens), broccoli (Brassica oleracea), and creeping bluegrass (Poa reptans). Along with lettuce, these plants represent several major crop families (Asteraceae, Brassicaceae, Fabaceae, Poaceae, and Solanaceae).

### Experimental Design

GFP-labeled X. autotrophicus was prepared as described above (Experiment 4).

Additionally, colonization was assessed on seedlings from surface sterilized seeds of lettuce (Lactuca sativa), tomato (Solanum lycopersicum), broccoli (Brassica oleracea), clover (Trifolium repens), and creeping bluegrass (Poa reptans). Seeds were surface sterilized as described above with adjustments to bleach concentration and exposure timing as follows: lettuce seeds as described above, tomato seeds were sterilized with 7.55% bleach for 50 minutes, broccoli seeds were sterilized using 50% bleach for 10 minutes, clover seeds were sterilized with 4% bleach for 1 hour, and creeping bluegrass were sterilized with 3% bleach for 1 hour. Seeds were then placed onto agarose plates, 8-10 seeds per plate, and spotted with GFP X. autotrophicus cultures (˜1e7 CFU per seed).

Seeds were germinated and grown under lab ambient conditions for 4-7 days to allow for early adequate root development for imaging. Seedlings were harvested and prepared for imaging as follows. First, seedling roots were treated with 0.13% DAB (3,3′-diaminobenzidine) mixed with 0.03% NBT (nitroblue tetrazolium) for 10 hours to neutralize ROS (Reactive Oxygen Species) in plant root cells. Roots were then stained with 0.01% Calcofluor White M2R (CFW) for 5 minutes before imaging to enhance visualization of root cells. Roots were imaged using an Olympus Fluoview FV1000 confocal laser scanning microscope with excitation at 405 nm and 488 nm for the CFW and GFP visualization, respectively.

### Results

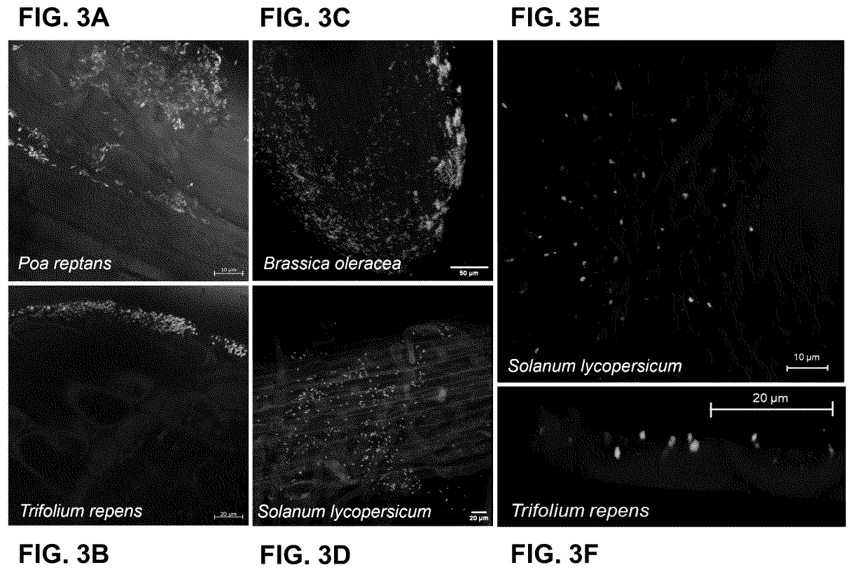

Across all assessed plant species, X. autotrophicus was consistently observed as an epiphyte, colonizing the root surface/rhizosphere (FIGS. 3A-3D). In some plants including clover and tomato, X. autotrophicus was also observed inside roots as a putative endophyte (FIGS. 3E-3F). These observations were achieved using a green fluorescent protein (GFP) expressing X. autotrophicus, following exposure of plant roots to reactive oxygen species (ROS) quenching molecules (3,3′-diaminobenzidine and nitroblue tetrazolium), suggesting X. autotrophicus may be sensitive to ROS produced inside plant cells. Collectively, these results highlight the potential of X. autotrophicus to interact with a wide range of plant species, an important characteristic of biofertilizers.

### Example 6. Primers for Lateral Root Development

The purpose of this experiment was to determine the efficacy of various sugars and organic acids as primers for enhancing lateral root development driven by microbial fertilizers.

### Experimental Design

Lateral root development was assessed in romaine lettuce seedlings (Lactuca sativa var. salivus, Johnny's Selected Seeds, Winslow, Maine, USA) as follows. 50% Hoagland's media prepared with 1.5% agar was adjusted to pH 6.5 and poured into 100 mm×100 mm square plates. Lettuce seeds were prepared for plating by first surface sterilizing and stratifying as follows: a 2 ml sterile microcentrifuge tube was filled with 500 μl of seeds along with 1 ml of 50% bleach. Seeds were exposed to bleach solution for 10 minutes after which the bleach solution was aspirated, and the seeds were washed with 1 ml of sterile water six times. Seeds were then resuspended 1 ml sterile water and placed at 4° C. for 48 hours to stratify after which the water was aspirated, and seeds were stored dry at 4° C. for up to 2 weeks before sowing. Sterile lettuce seeds were placed near the top of the 50% Hoagland's agar plate along the “sowing line”. Using a pipette, 30 μl of either water (negative control) or tested product (as disclosed in Table 1), were plated in a single line 45 mm below the sowing line. Plates were then placed in a growth chamber with 16-hour day/8-hour night light cycles, temperatures at 25° C. during the day and 22° C. at night, and humidity maintained at 50%. Plates were kept at a ˜80° angle during growth to encourage root growth along the agar surface. Seedlings were grown for 7 days and then root development was assessed as lateral root density determined by dividing the lateral root number of total tap root length.

TABLE 1
Lateral root experimental treatments

| Treat- | | |
|---|---|---|
| ment | | |
| # | Treatment Description | Primer Tested |
| 1 | Negative control (Water Only) | — |
| 2 | X. autotrophicus/diluent (X. autotrophicus | — |
| | 7c grown in a bioreactor and diluted | |
| | in a diluent produced using 21.1 g/L | |
| | of alfalfa extract) | |
| 3 | 5% (w/v) sucrose | +5% (w/v) sucrose |
| 4 | X. autotrophicus/diluent + | +5% (w/v) sucrose |
| | 5% (w/v) sucrose | |
| 5 | 5% (w/v) malic acid | +5% (w/v) malic |
| | | acid |
| 6 | X. autotrophicus/diluent + | +5% (w/v) malic |
| | 5% (w/v) malic acid | acid |
| 7 | 5% (w/v) fructose | +5% (w/v) fructose |
| 8 | X. autotrophicus/diluent + | +5% (w/v) fructose |
| | 5% (w/v) fructose | |
| 9 | 5% (w/v) glucose | +5% (w/v) glucose |
| 10 | X. autotrophicus/diluent + | +5% (w/v) glucose |
| | 5% (w/v) glucose | |

### Results

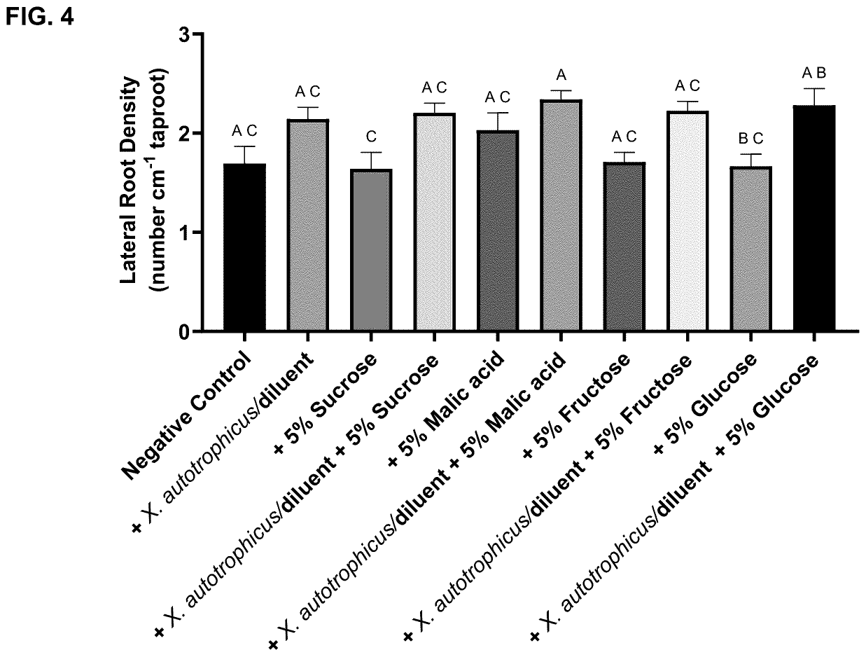

Application of X. autotrophicus/diluent alone increased lateral root density of lettuce seedlings compared to the negative control (FIG. 4). This expected result validated the outcomes of this assay.

When used as a primer for X. autotrophicus/diluent, 5% (w/v) malic acid and 5% (w/v) glucose resulted in increased lateral root density compared to X. autotrophicus/diluent alone (FIG. 4). Of the tested sugars and organic acids, only 5% (w/v) malic acid increased lateral root density when applied alone (FIG. 4).

Together, these results indicate that malic acid and glucose can each serve as effective primers for enhancing lateral root development driven by microbial fertilizers.

### Example 7. Use of Sucrose as a X. autotrophicus Primer when Applied to Corn

The purpose of this experiment was to test the use of sucrose as a primer of X. autotrophicus/diluent when applied to corn as a seed coat.

### Experimental Design

Corn seed (hybrid variety: RU46-02 MC 52.8 37902) were sown in 6″ pots containing sunshine mix #15. Seeds were either received X. autotrophicus/diluent (X. autotrophicus 7c grown in bioreactor culture and diluted in diluent produced using 21.1 g/L alfalfa extract) at a seed coat at planting or were uncoated. Additionally, seeds received either 0.193 ml of sterile tap water or 0.193 ml of 5% (w/v) sucrose solution according to treatments as outlined in Table 2. Corn plants were grown to V6 stage (˜28 days after sowing) in a growth chamber with 16-hour day/8-hour night light cycles, temperatures at 25° C. during the day and 22° C. at night, and humidity maintained at 50%. During the growth period, plants received weekly doses of nitrogen fertilizer as AN-20 and nitrogen free Hoagland's solution for micronutrients. All plants received nitrogen fertilizer at 100% GSP (equivalent to 168 lbs. N per acre). Plants were watered through an automatic drip irrigation system that watered daily during the growth period. At V6, plants were harvested for aboveground fresh biomass, aboveground dry biomass, and leaf tissue nitrogen (N).

TABLE 2
Example 7 Treatment Groups

| | | | X. | |
|---|---|---|---|---|
| Treatment | Treatment | | autotrophicus/ | |
| # | Description | Nitrogen | diluent | Primer |
| 1 | Untreated Control | 100% GSP | — | — |
| 2 | +X. autotrophicus/ | | ✓ | — |
| | diluent | | | |
| 3 | +X. autotrophicus/ | | ✓ | ✓ |
| | diluent + | | | |
| | 5% (w/v) sucrose | | | |

### Results

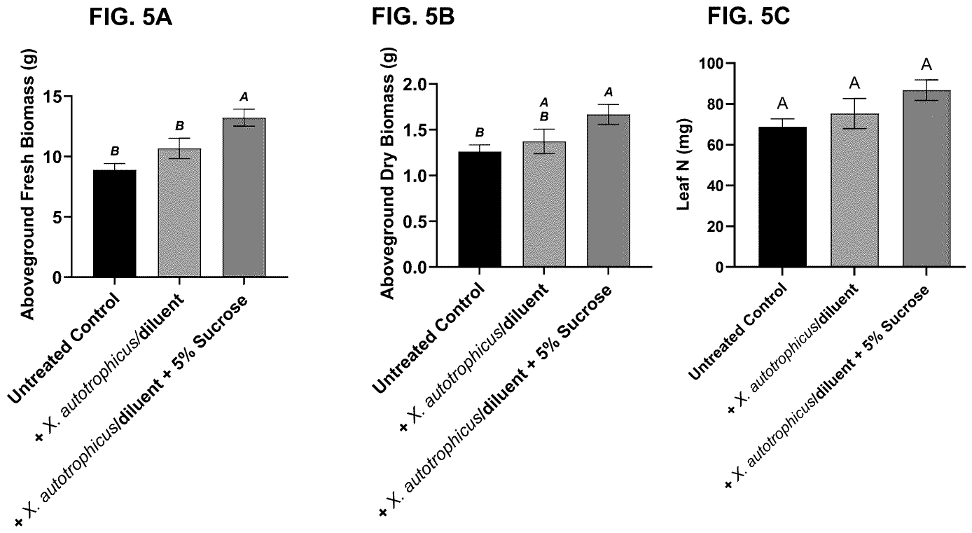

Application of X. autotrophicus/diluent to corn seed as a seed coating boosted aboveground fresh biomass by nearly 20% (FIG. 5A) and total leaf nitrogen by over 9% (FIG. 5C).

When primed with 5% sucrose, X. autotrophicus/diluent efficacy was enhanced by approximately 3-fold leading to a 48.7% increase in aboveground fresh biomass, 32.3% increase in aboveground dry biomass, and 26.1% increase in leaf nitrogen (Table 3, FIGS. 5A-5C).

TABLE 3
Relative percent increase in measured variables
with and without sucrose priming

| | % Increase over Control | | |
|---|---|---|---|
| | Aboveground | Aboveground | Leaf |
| | fresh weight | dry weight | Nitrogen |
| Treatment | (g) | (g) | (mg) |
| X. autotrophicus/diluent | +19.9% | +8.8% | +9.4% |
| X. autotrophicus/ | +48.7% | +32.3% | +26.1% |
| diluent + 5% sucrose | | | |
| Primer Effect | 2.4x | 3.6x | 2.8x |

The above results indicate that sucrose served effectively as an X. autotrophicus primer when applied to corn as a seed coat.

### Example 8. Use of Sucrose as a X. autotrophicus Primer when Applied to Lettuce

The purpose of this trial was to evaluate the use of sucrose as a primer of X. autotrophicus when applied to lettuce.

### Experimental Design

Romaine lettuce seedlings (Lactuca sativa var. salivus, Johnny's Selected Seeds, Winslow, Maine, USA) were sown into trays containing sunshine mix #1. Once germinated, seedlings were watered as needed and provided weekly doses of 50% Hoagland's solution with nitrogen to support growth. Seedlings were grown for 21-days before transplanting for the experimental trial. Seedlings were transplanted into 4″ pots containing sunshine mix #15 potting media. Lettuce plants were grown for 28 days from transplant to harvest in a growth chamber with 16-hour day/8-hour night light cycles, temperatures at 25° C. during the day and 22° C. at night, and humidity maintained at 50%. During the growth period, plants received weekly doses of nitrogen fertilizer as UAN-32 and nitrogen free Hoagland's solution for micronutrients. Nitrogen fertilizer was applied at 80% grower standard practice (GSP) or 100% GSP according to the treatments listed in Table 4 or Table 5 below. Plants were watered through an automatic drip irrigation system that watered daily during the growth period. X. autotrophicus diluent or sucrose-primed X. autotrophicus diluent was applied as a drench during weekly applications. X. autotrophicus diluent was primed using 2.5%, 5%, or 10% (w/v) sucrose. Sucrose was added to liquid X. autotrophicus diluent and then incubated for 2 hours at room temperature before applying to plants. After 28 days of growth, lettuce plants are harvested for aboveground fresh biomass, aboveground dry biomass, and leaf tissue nitrogen (N).

TABLE 4
Treatment Groups

| | | | X. | |
|---|---|---|---|---|
| Treatment | Treatment | | autotrophicus/ | |
| # | Description | Nitrogen | diluent | Primer |
| 1 | 80% GSP | 38 lb. N/ac | — | — |
| 2 | +X. autotrophicus/ | | ✓ | — |
| | diluent | | | |
| 3 | +X. autotrophicus/ | | ✓ | ✓ |
| | diluent + | | | |
| | 5% (w/v) sucrose | | | |
| 4 | 100% GSP | 48 lb. N/ac | — | — |

TABLE 5
Expanded Treatment Groups

| | | | X. | |
|---|---|---|---|---|
| Treatment | Treatment | | autotrophicus/ | |
| # | Description | Nitrogen | diluent | Primer |
| 1 | 80% GSP | 38 lb. N/ac | — | — |
| 2 | +2.5% (w/v) sucrose | | — | ✓ |
| 3 | +5% (w/v) sucrose | | — | ✓ |
| 4 | +10% (w/v) sucrose | | — | ✓ |
| 5 | +X. autotrophicus/ | | ✓ | — |
| | diluent | | | |
| 6 | +X. autotrophicus/ | | ✓ | ✓ |
| | diluent + | | | |
| | 2.5% (w/v) sucrose | | | |
| 7 | +X. autotrophicus/ | | ✓ | ✓ |
| | diluent + 5% | | | |
| | (w/v) sucrose | | | |
| 8 | +X. autotrophicus/ | | ✓ | ✓ |
| | diluent + | | | |
| | 10% (w/v) sucrose | | | |
| 9 | 100% GSP | 48 lb. N/ac | — | — |

### Results

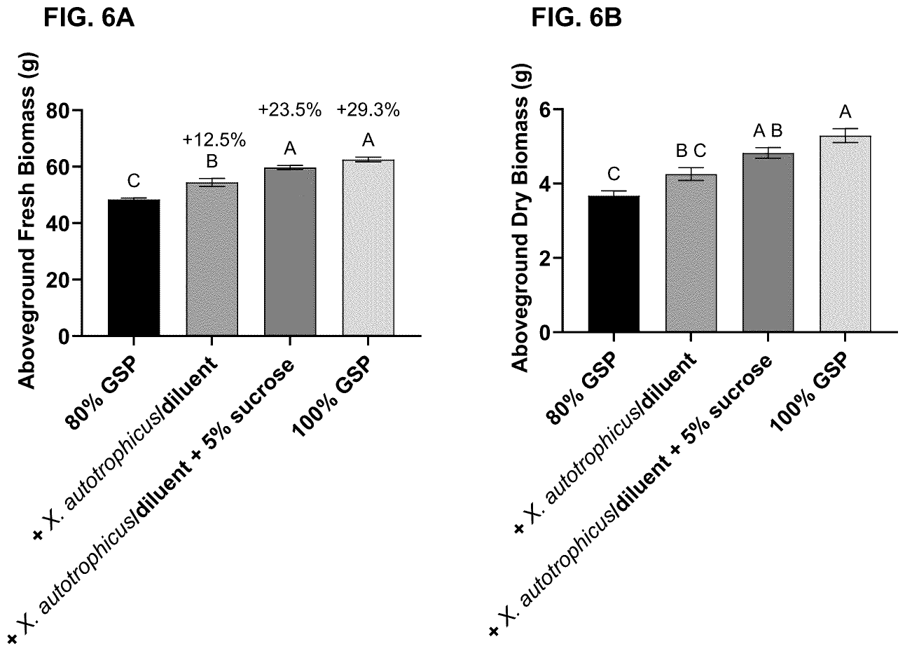

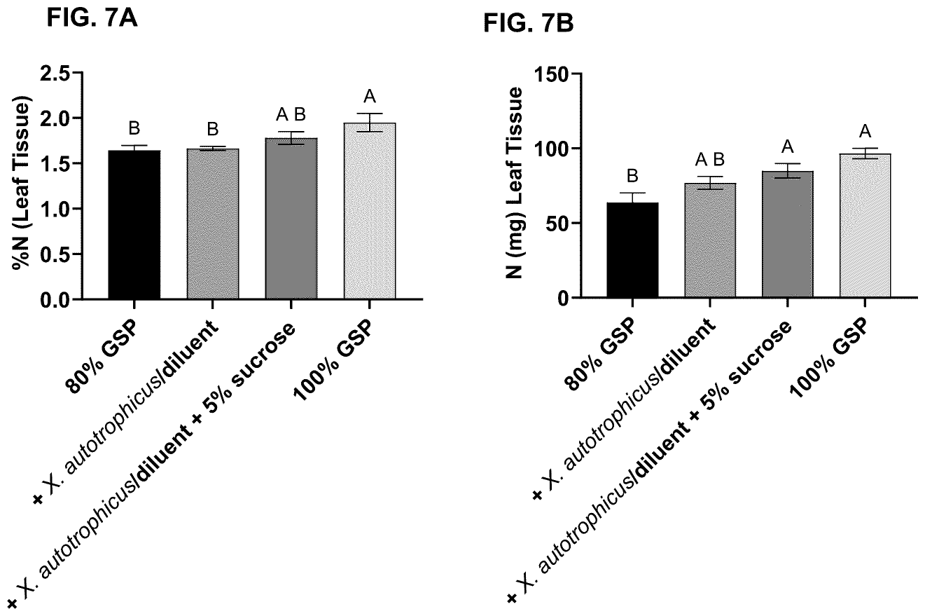

Application of X. autotrophicus to lettuce at 80% GSP increased yield by 12.5% compared to the 80% GSP control (FIG. 6A). When X. autotrophicus was primed with 5% sucrose before application, product efficacy was increased by approximately 3-fold (FIG. 6A). Primed X. autotrophicus increased yield by 23.5% (1.9× over X. autotrophicus) compared to the 80% GSP control and statistically matched the 100% GSP treatment (FIG. 6A). Primed X. autotrophicus also statistically matched the 100% GSP treatment in aboveground dry biomass (2.9× over X. autotrophicus) (FIG. 6B), % N in leaf tissue (7× over X. autotrophicus) (FIG. 7A), and total N in leaf tissue (1.6× over X. autotrophicus) (FIG. 7B).

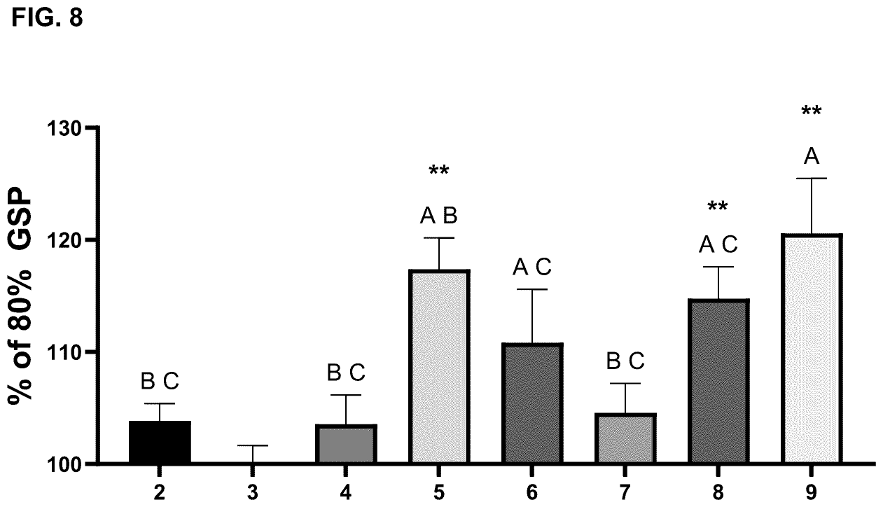

The addition of sucrose (2.5%-10%) to plants without X. autotrophicus did not significantly increase yield of lettuce, thus supporting that priming effects predominately impact microbial activity (FIG. 8). When X. autotrophicus was applied without a primer, lettuce yield was significantly increased compared to the 80% GSP control with an average increase of 17.4% (FIG. 8). Addition of sucrose as a primer to X. autotrophicus, irrespective of concentration, did not enhance efficacy of X. autotrophicus in this trial.

Collectively, the above results indicate that sucrose served effectively as an X. autotrophicus primer when applied to lettuce.

### Example 9. Efficacy of Sucrose and Malic Acid as X. autotrophicus Primers

The purpose of this trial was to test the use of sucrose and malic acid at various concentrations as primers of X. autotrophicus when applied to lettuce.

### Experimental Design

Romaine lettuce seedlings (Lactuca sativa var. salivus, Johnny's Selected Seeds, Winslow, Maine, USA) were sown into trays containing sunshine mix #1. Once germinated, seedlings were watered as needed and provided weekly doses of 50% Hoagland's solution with nitrogen to support growth. Seedlings were grown for 21-days before transplanting for the experimental trial. Seedlings were transplanted into 4″ pots containing sunshine mix #15 potting media. Lettuce plants were grown for 28 days from transplant to harvest in a growth chamber with 16-hour day/8-hour night light cycles, temperatures at 25° C. during the day and 22° C. at night, and humidity maintained at 50%. During the growth period, plants received weekly doses of nitrogen fertilizer as UAN-32 and nitrogen free Hoagland's solution for micronutrients. Nitrogen fertilizer was applied at 80% grower standard practice (GSP) or 100% GSP according to the treatments listed in Table 6 below. Plants were watered through an automatic drip irrigation system that watered daily during the growth period. Freeze-dried X. autotrophicus (X. autotrophicus 7c grown in bioreactor culture and freeze-dried as a concentrated powder) or primed Freeze-dried X. autotrophicus were applied as a drench during weekly applications. Freeze-dried X. autotrophicus was primed using 2.5%, 5%, or 10% (w/v) sucrose or malic acid. Solutions of sucrose or malic acid (10 ml) were added to 147.93 mg of freeze-dried X. autotrophicus and then incubated for 2 hours at room temperature before applying to plants. After 28 days of growth, lettuce plants are harvested for aboveground fresh biomass.

TABLE 6
Example 9 Treatment Groups

| Treatment | | |
|---|---|---|
| # | Treatment Description | Nitrogen |
| 1 | 80% GSP | 38 lb. N/ac |
| 2 | +Freeze-dried X. autotrophicus | |
| 3 | +Freeze-dried X. autotrophicus + | |
| | 2.5% (w/v) sucrose | |
| 4 | +Freeze-dried X. autotrophicus + | |
| | 5% (w/v) sucrose | |
| 5 | +Freeze-dried X. autotrophicus + | |
| | 10% (w/v) sucrose | |
| 6 | +Freeze-dried X. autotrophicus + | |
| | 2.5% (w/v) malic acid | |
| 7 | +Freeze-dried X. autotrophicus + | |
| | 5% (w/v) malic acid | |
| 8 | +Freeze-dried X. autotrophicus + | |
| | 10% (w/v) malic acid | |
| 9 | 100% GSP | 48 lb. N/ac |

### Results

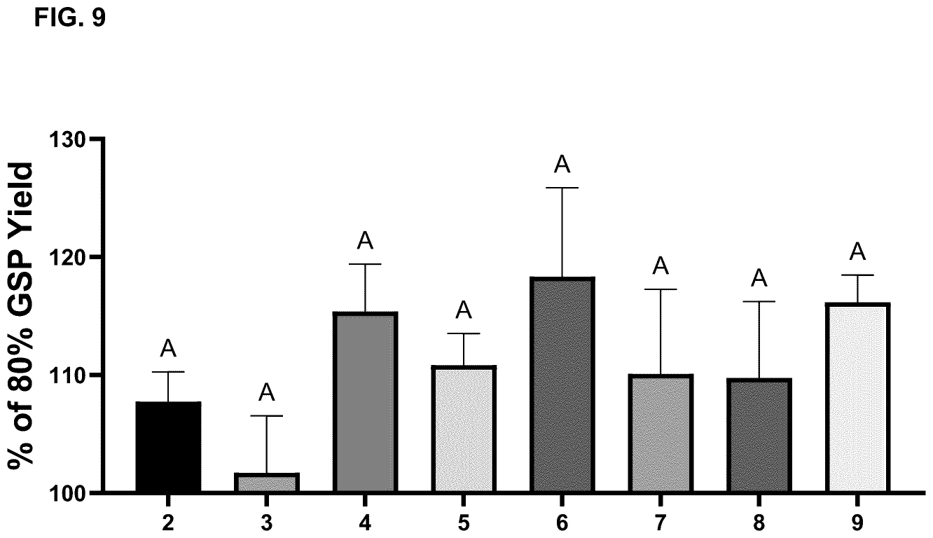

This experiment investigated the potential for sucrose and malic acid to act as primers for freeze-dried X. autotrophicus. No statistically significant impact on the efficacy of freeze-dried X. autotrophicus was observed when adding sucrose or malic acid as primers (FIG. 9). However, priming with 5% (w/v) sucrose or 2.5% (w/v) malic acid did result in greater yield increases over 80% GSP (FIG. 9).

### Example 10: Efficacy of Malic Acid, Glutamic Acid, and Pantothenic Acid as X. autotrophicus Primers in Lettuce

The purpose of this trial was to evaluate the use of malic acid, glutamic acid, and pantothenic acid as primers for freeze-dried X. autotrophicus when applied to lettuce.

### Experimental Design

Romaine lettuce seedlings (Lactuca sativa var. salivus, Johnny's Selected Seeds, Winslow, Maine, USA) were sown into trays containing sunshine mix #1. Once germinated, seedlings were watered as needed and provided weekly doses of 50% Hoagland's solution with nitrogen to support growth. Seedlings were grown for 21-days before transplanting for the experimental trial. Seedlings were transplanted into 4″ pots containing sunshine mix #15 potting media. Lettuce plants were grown for 28 days from transplant to harvest in a growth chamber with 16-hour day/8-hour night light cycles, temperatures at 25° C. during the day and 22° C. at night, and humidity maintained at 50%. During the growth period, plants received weekly doses of nitrogen fertilizer as UAN-32 and nitrogen free Hoagland's solution for micronutrients. Nitrogen fertilizer was applied at 80% GSP or 100% GSP according to the treatments listed in Table 7 below. Plants were watered through an automatic drip irrigation system that watered daily during the growth period. freeze-dried X. autotrophicus or primed freeze-dried X. autotrophicus was applied as a drench during weekly applications according to Table 7 below. Solutions of primers were prepared according to Table 7 below, then 5 ml of each solution were added to 88.76 mg of freeze-dried X. autotrophicus and incubated for 2 hours at room temperature before applying to plants. After 28 days of growth, lettuce plants are harvested for aboveground fresh biomass measurement.

TABLE 7
Example 10 Treatment Groups

| Treatment | | |
|---|---|---|
| # | Treatment Description | Nitrogen |
| 1 | 80% GSP | 38 lb. N/ac |
| 2 | Freeze-dried X. autotrophicus | |
| 3 | 2.5% (w/v) malic acid | |
| 4 | Freeze-dried X. autotrophicus + | |
| | 2.5% (w/v) malic acid | |
| 5 | 1.0M glutamic acid | |
| 6 | Freeze-dried X. autotrophicus + | |
| | 1.0M glutamic acid | |
| 7 | 21.5 μg/mL pantothenic acid | |
| 8 | Freeze-dried X. autotrophicus + | |
| | 21.5 μg/mL pantothenic acid | |
| 9 | 100% GSP | 48 lb. N/ac |

### Results

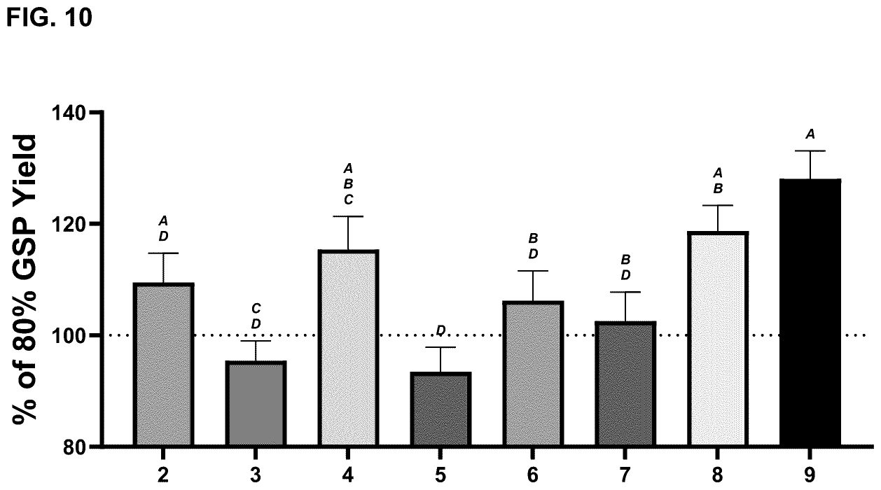

This experiment investigated the potential for malic acid, glutamic acid, and pantothenic acid to act as primers for freeze-dried X. autotrophicus. No statistically significant impact on the overall efficacy of freeze-dried X. autotrophicus was found using malic acid, glutamic acid, or pantothenic acid as primers. (FIG. 10). However, priming with 2.5% (w/v) malic acid or 21.5 μg/mL pantothenic acid resulted in greater yield increases over 80% GSP than the application of X. autotrophicus alone (FIG. 10). Additionally, the addition of primers alone (without X. autotrophicus) did not enhance yield, indicating these primers function primarily to enhance X. autotrophicus efficacy.

### Example 11: Efficacy of Food Waste Powders as Primers for X. autotrophicus

The purpose of this experiment was to evaluate the use of food waste powders or sugars (sucrose and fructose) as primers for freeze-dried X. autotrophicus nitrogen fixation (N-fixation) activity.

### Experimental Design

Food waste powders were tested as complex sources of sugars and amino acids. N-fixation activity was measured in this experiment using a colorimetric assay for measuring pH change during N-fixation (hereafter referred to as the Nitrogen Fixation Colorimetric Assay, NFCA). Briefly, tested microbial cells were pelleted and washed of any media. Cells were then resuspended in nitrogen-free growth media (pH=6.5) supplemented with bromothymol blue pH indicator to a cell density of 1.0e8 CFU ml⁻¹. Samples were incubated at 30° C. in a static incubator and measured every 24 hours beginning at time 0 for up to 7 days for absorbance of bromothymol blue (616 nm) using a 96-well plate compatible spectrophotometer. Raw absorbance values were background corrected using an uninoculated control sample. N-fixation rates were then estimated by calculating the change in absorbance over time. In this experiment, primers were tested during rehydration of X. autotrophicus product. Tested primers (10 mg each) were added to 100 mg of freeze-dried X. autotrophicus and then rehydrated with 1 ml of DI water. Samples were incubated at room temperature for 2 hours with intermittent mixing every 30 minutes. After 2 hours, samples were pelleted and washed of media before performing the colorimetric assay as described above.

TABLE 8
Example 11 Treatment Groups

| | | Primer | |
|---|---|---|---|
| Treatment | | Concentration | Primer |
| # | Treatment Description | (mg/ml) | effect |
| 1 | Freeze-dried X. autotrophicus | — | — |
| | (Control) | | |
| 2 | +Tomato powder | 10 | Null |
| 3 | +Corn powder | 10 | Null |
| 4 | +Sucrose | 10 | Null |
| 5 | +Fructose | 10 | Null |

### Results

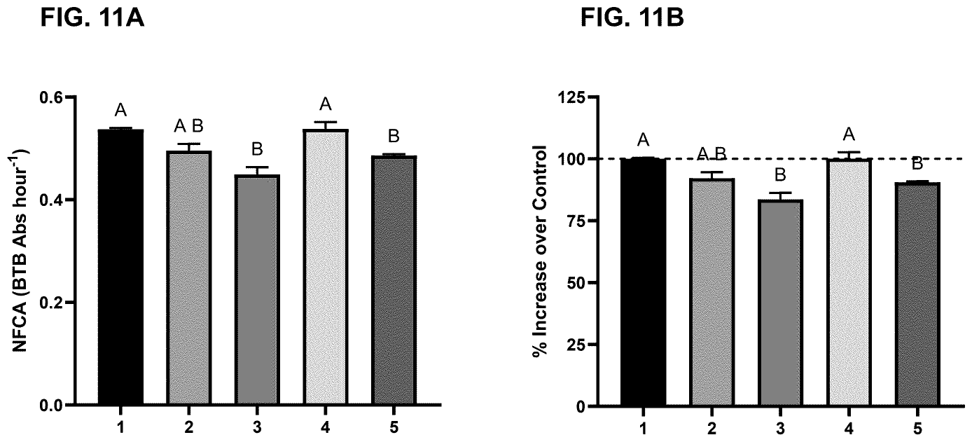

This experiment investigated the potential of tomato powder, corn powder, sucrose, and fructose to prime the N-fixation activity of freeze-dried X. autotrophicus when added during rehydration of the dried product. None of the tested primers resulted in a change in bromothymol blue absorbance over time (FIG. 11A) or a percent increase in nitrogen fixation activity (FIG. 11B).

### Example 12: Efficacy of Amino Acids as Primers for X. autotrophicus

The purpose of this experiment was to evaluate the use of amino acids as primers for freeze-dried X. autotrophicus N-fixation activity.

### Experimental Design

For Examples 13-19, N-fixation activity was following the standard NFCA protocol. Briefly, tested microbial cells are pelleted and washed of any media. Cells were then resuspended in nitrogen-free growth media (pH=6.5) supplemented with bromothymol blue pH indicator to a cell density of 1.0e8 CFU ml⁻¹. Samples were incubated at 30° C. in a static incubator and measured every 24 hours beginning at time 0 for up to 7 days for absorbance of bromothymol blue (616 nm) using a 96-well plate compatible spectrophotometer. Raw absorbance values were background corrected using an uninoculated control sample. N-fixation rates are then estimated by calculating the change in absorbance over time.

In this experiment, primers were tested during rehydration of freeze-dried X. autotrophicus. Solutions shown in Table 9, including five individual amino acids and a mixture of all amino acids (amounts of each equivalent to amounts tested of individual amino acids) were prepared and then 1 ml of each solution was added to 100 mg of freeze-dried X. autotrophicus. Samples were incubated at room temperature for 2 hours with intermittent mixing every 30 minutes. After 2 hours, samples were pelleted and washed of media before performing the colorimetric assay as described above.

TABLE 9
Example 12 Treatment Groups

| | | Primer | |
|---|---|---|---|
| Treatment | | Concentration | Primer |
| # | Treatment Description | (% w/v) | effect |
| 1 | Freeze-dried X. autotrophicus | — | — |
| | (Control) | | |
| 2 | +Arginine | 2.8 | 1.1x |
| 3 | +Asparagine | 0.6 | 1.6x |
| 4 | +Glutamic acid | 14.6 | 2.4x |
| 5 | +Glycine | 2.3 | 3.6x |
| 6 | +Tryptophan | 0.7 | 3.2x |
| 7 | +Amino acid mix | 21 | 2.7x |

### Results

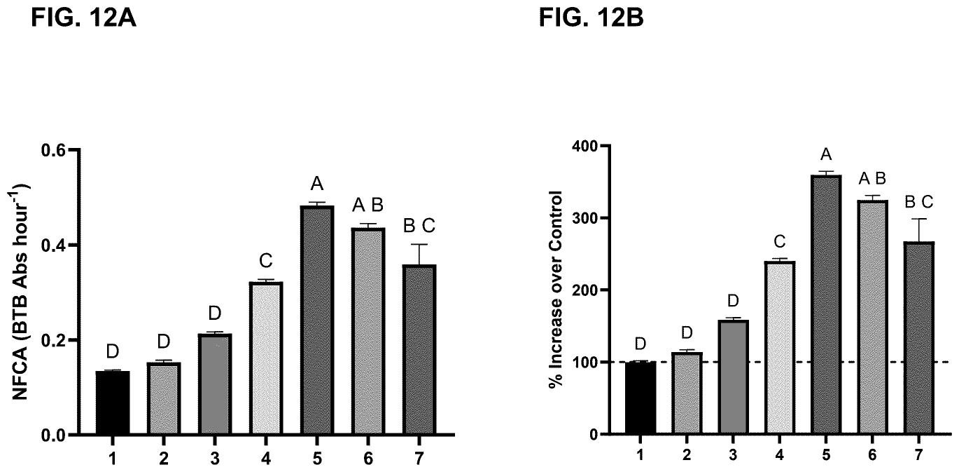

This experiment investigated the potential of amino acids including arginine, asparagine, glutamic acid, glycine, tryptophan and a mixture of these amino acids, to prime the N-fixation activity of freeze-dried X. autotrophicus when added during rehydration of the dried product. All tested amino acids resulted in an increase in bromothymol blue absorbance over time (FIG. 12A) and a percent increase in nitrogen fixation activity (FIG. 12B). Of these, glutamic acid, glycine, tryptophan, and the amino acid mixture increased N-fixation of freeze-dried X. autotrophicus by 2.4×-3.6× (Table 9).

### Example 13: Efficacy of Salts and Nutrient Broth as Primers for X. autotrophicus

The purpose of this trial was to evaluate the use of salt solutions as primers for supporting improved rehydration recovery of freeze-dried X. autotrophicus and thereby enhancing N-fixation activity. Additionally, nutrient broth (NB) medium was also tested as a primer. NB is both a source of amino acids and, as a standard growth medium, was hypothesized to support improved rehydration recovery of freeze-dried X. autotrophicus and thereby enhance N-fixation activity.

### Experimental Design

In this experiment, primers were tested during rehydration of freeze-dried X. autotrophicus. 50 mg/mL of sodium chloride, potassium chloride, calcium chloride, sodium bicarbonate, or nutrient broth were added to 100 mg of freeze-dried X autotrophicus and then rehydrated with 1 ml of DI water (Table 10). Samples were incubated at room temperature for 2 hours with intermittent mixing every 30 minutes. After 2 hours, samples were pelleted and washed of media before performing the colorimetric assay as described above.

TABLE 10
Example 13 Treatment Groups

| | | Primer | |
|---|---|---|---|
| Treatment | | Concentration | Primer |
| # | Treatment Description | (mg/ml) | effect |
| 1 | Freeze-dried X. autotrophicus | — | — |
| | (Control) | | |
| 2 | +Sodium chloride | 50 | Null |
| 3 | +Potassium chloride | 50 | 1.2x |
| 4 | +Calcium chloride | 50 | Null |
| 5 | +Sodium Bicarbonate | 50 | Null |
| 6 | +Nutrient Broth | 50 | Null |

### Results

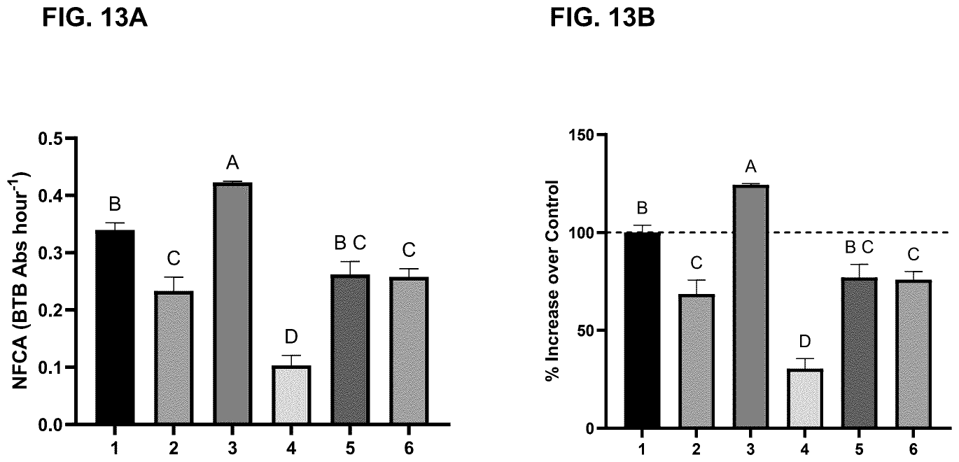

This experiment investigated whether salts or NB media would serve effectively as primers to support enhanced N-fixation activity of freeze-dried X. autotrophicus. Of the tested primers, only potassium chloride significantly increased bromothymol blue absorbance over time (1.2× the rate of the control) (FIG. 13A) and resulted in a significant percent increase in nitrogen fixation activity (FIG. 13B).

### Example 14: Effect of Concentration on Amino Acid Primer Efficacy

The purpose of this experiment was to evaluate the use of glycine, glutamic acid, histidine, and arginine at two different concentrations as primers for freeze-dried X. autotrophicus N-fixation activity.

### Experimental Design

In this experiment, primers were tested during rehydration of freeze-dried X. autotrophicus. Solutions of tested amino acids shown in Table 11 were prepared and then 1 ml of each solution was added to 100 mg of freeze-dried X. autotrophicus. Samples were incubated at room temperature for 2 hours with intermittent mixing every 30 minutes. After 2 hours, samples were pelleted and washed of media before performing the colorimetric assay as described above.

TABLE 11
Example 14 Treatment Groups

| | | Primer | |
|---|---|---|---|
| Treatment | | Concentration | Primer |
| # | Treatment Description | (M) | effect |
| 1 | Freeze-dried X. autotrophicus | — | — |
| 2 | +Glycine | 0.03 | 1.9x |
| 3 | | 1.0 | 2.5x |
| 4 | +Glutamic acid | 0.03 | 10.6x |
| 5 | | 1.0 | 15.9x |
| 6 | +Histidine | 0.03 | 11.4x |
| 7 | | 1.0 | 9.1x |
| 8 | +Arginine | 0.03 | Null |
| 9 | | 1.0 | 1.6x |

### Results

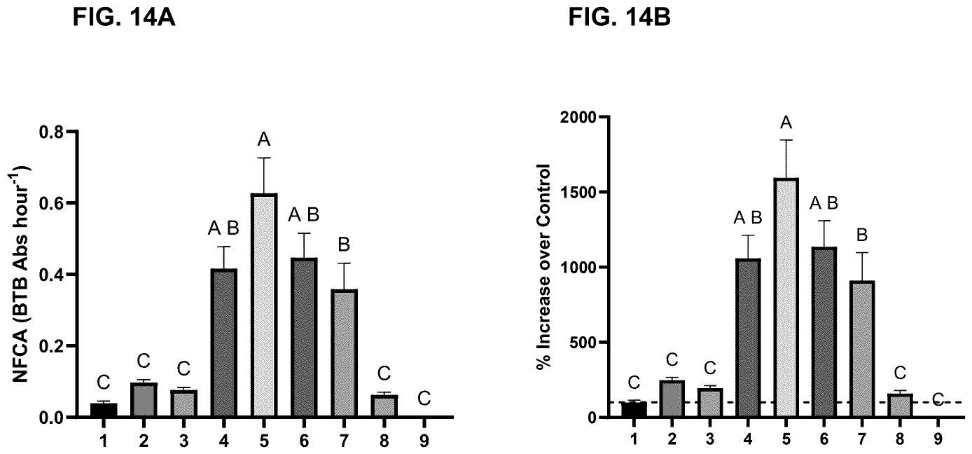

This experiment investigated several amino acids for the potential to prime N-fixation activity of freeze-dried X. autotrophicus when added during rehydration. Glutamic acid and histidine as primers at 0.03M and 1.0M significantly increased the N-fixation activity of freeze-dried X. autotrophicus (FIGS. 14A-14B). Glutamic acid increased freeze-dried X. autotrophicus N-fixation by 10.6× when applied at 0.03M and 15.9× when applied at 1.0M (FIGS. 14A-14B). Histidine increased freeze-dried X. autotrophicus N-fixation by 10.6× when applied at 0.03M and 15.9× when applied at 1.0M (FIGS. 14A-14B). Glycine also measurably increased N-fixation activity of freeze-dried X. autotrophicus by 1.9× and 2.5× when applied at 0.03M and 1.OM, respectively, however this increase was not statistically significant (FIGS. 14A-14B). Arginine did not increase N-fixation activity of freeze-dried X. autotrophicus when applied at the lower 0.03M concentration, but did measurably, though not statistically significantly, increase N-fixation by 1.6× when applied at 1.OM. (FIGS. 14A-14B).

### Example 15: Efficacy of Thiamine and Pantothenic Acid as X. autotrophicus Primers

The purpose of this trial was to evaluate the use of Vitamin B forms including thiamine and pantothenic acid as primers for freeze-dried X. autotrophicus N-fixation activity.

### Experimental Design

In this experiment, primers were tested during rehydration of freeze-dried X. autotrophicus. Solutions of thiamine and pantothenic acid shown in Table 12 were prepared and 1 ml was added to 100 mg of freeze-dried X. autotrophicus. Additionally, a mixture of thiamine and pantothenic acid was also tested as a primer. Samples were incubated at room temperature for 2 hours with intermittent mixing every 30 minutes. After 2 hours, samples were pelleted and washed of media before performing the colorimetric assay as described above.

TABLE 12
Example 15 Treatment Groups

| | | Primer | |
|---|---|---|---|
| Treatment | | Concentration | Primer |
| # | Treatment Description | (ug/ml) | effect |
| 1 | Freeze-dried X. | — | — |
| | autotrophicus (Control) | | |
| 2 | Thiamine | 0.3 | Null |
| 3 | | 0.7 | Null |
| 4 | | 3.3 | 1.2x |
| 5 | | 6.7 | 1.2x |
| 6 | Pantothenic acid | 2.2 | 1.4x |
| 7 | | 4.3 | 1.1x |
| 8 | | 21.5 | 1.5x |
| 9 | | 43.0 | Null |
| 10 | Thiamine + Pantothenic acid | 0.7 thiamine + 4.3 | 1.2x |
| | | pantothenic acid | |

### Results

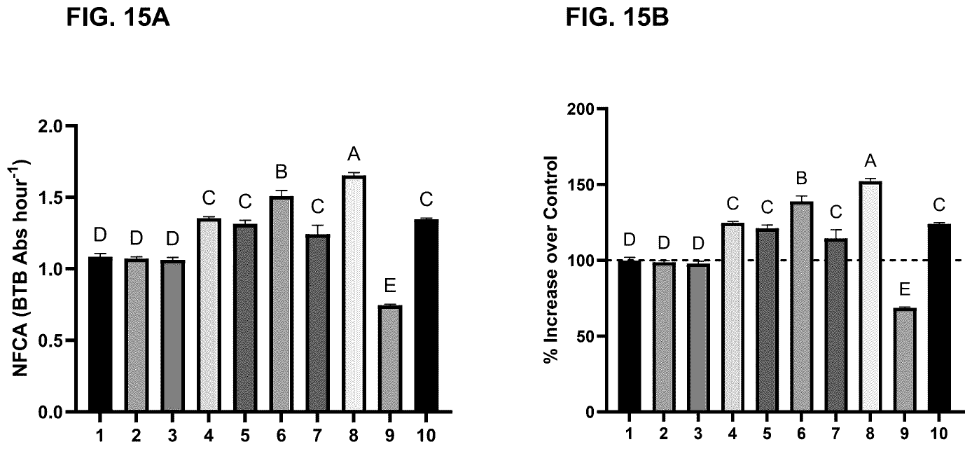

This experiment investigated the use of thiamine and pantothenic acid as primers for N-fixation activity of freeze-dried X. autotrophicus. Thiamine significantly increased N-fixation activity of freeze-dried X. autotrophicus when added at >3.3 μg/ml (FIGS. 15A-15B). Pantothenic acid significantly increased N-fixation activity of freeze-dried X. autotrophicus when added between 2.2 and 21.5 μg/ml (FIGS. 15A-15B). A mixture of thiamine (0.7 μg/ml) and pantothenic acid (4.3 μg/ml) also significantly increased N-fixation activity of freeze-dried X. autotrophicus (FIGS. 15A-15B).

### Example 16: Efficacy of Molybdenum and Iron as Primers for X. Autotrophicus

The purpose of this trial was to test the use of bioavailable forms of molybdenum (sodium molybdate) and iron (iron EDTA) as primers for X. autotrophicus/diluent N-fixation activity.

### Experimental Design

N-fixation activity was measured using the standard NFCA protocol. Briefly, tested microbial cells were pelleted and washed of any media. Cells were then resuspended in nitrogen-free growth media (pH=6.5) supplemented with bromothymol blue pH indicator to a cell density of 1.0e8 CFU ml⁻¹. To test priming effect of sodium molybdate and iron EDTA, N-free media was supplemented with solutions of these compounds as detailed in Table 13. The standard N-free media contained 0.008 mM sodium molybdate and 0.179 mM iron EDTA. Priming with these compounds was tested at 2×, 5×, and 10× these standard concentrations. Samples were incubated at 30° C. in a static incubator and measured every 24 hours beginning at time 0 for up to 7 days for absorbance of bromothymol blue (616 nm) using a 96-well plate compatible spectrophotometer. Raw absorbance values were background corrected using an uninoculated control sample. N-fixation rates were then estimated by calculating the change in absorbance over time.

TABLE 13
Example 16 Treatment Groups

| | | Primer | |
|---|---|---|---|
| | | Concentration | Primer |
| Treatment # | Treatment Description | (mM) | effect |
| 1 | X. autotrophicus/diluent | — | — |
| | (Control) | | |
| 2 | +Sodium Molybdate | 0.016 | Null |
| 3 | | 0.064 | Null |
| 4 | | 0.080 | 1.1x |
| 5 | +Iron EDTA | 0.358 | Null |
| 6 | | 0.895 | Null |
| 7 | | 1.790 | Null |

TABLE 14
Expanded Example 16 Treatment Groups

| | | Primer | |
|---|---|---|---|
| | | Concentration | Primer |
| Treatment # | Treatment Description | (mM) | effect |
| 1 | X. autotrophicus/diluent | — | — |
| | (Control) | | |
| 2 | +Sodium Molybdate | 0.016 | 1.3x |
| 3 | | 0.064 | 1.3x |
| 4 | | 0.080 | 1.3x |
| 5 | +Iron EDTA | 0.358 | Null |
| 6 | | 0.895 | 1.1x |
| 7 | | 1.790 | 1.1x |

### Results

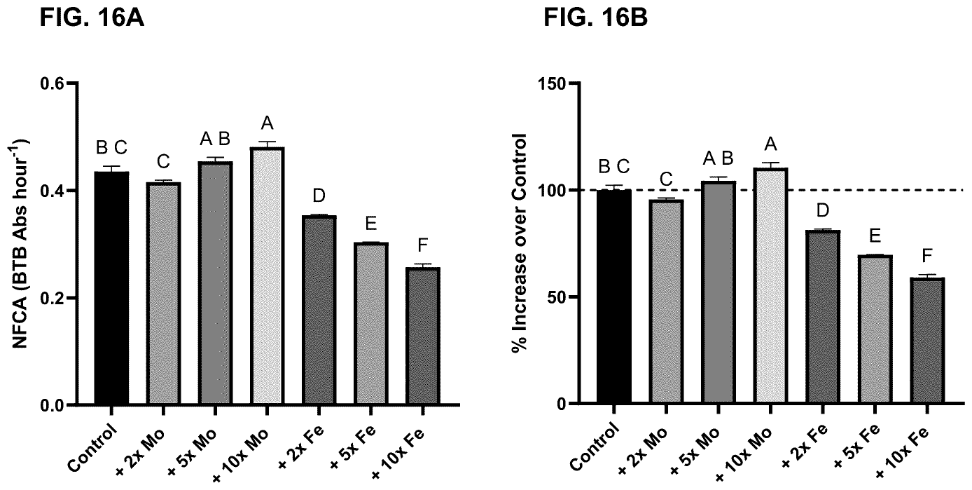

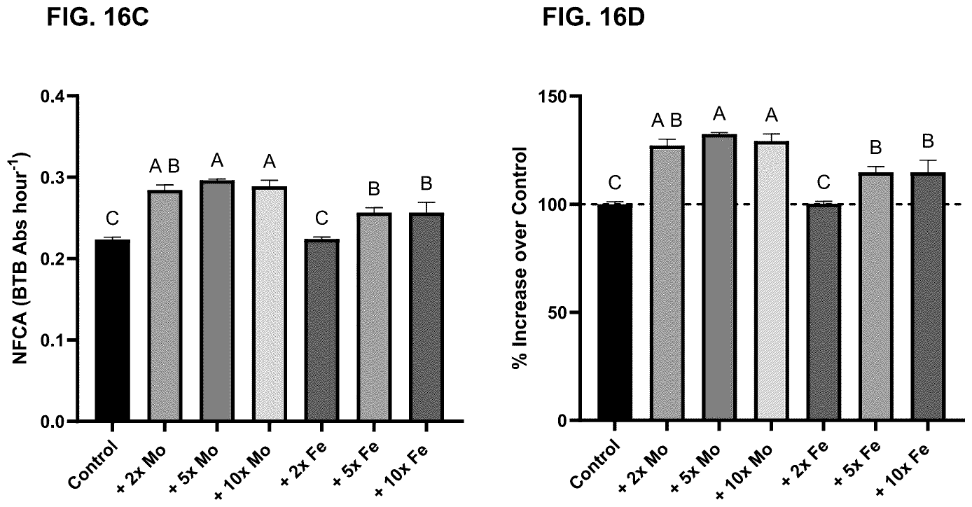

This experiment investigated use of sodium molybdate and iron EDTA as primers for X. autotrophicus/diluent. Sodium molybdate at 10× concentration (0.080 mM) significantly increased bromothymol blue absorbance over time (FIG. 16A) and resulted in a significant percent increase in nitrogen fixation activity (FIG. 16B).

This experiment investigated the use of sodium molybdate and iron EDTA as primers for X. autotrophicus. Sodium molybdate at all concentrations significantly increased increased bromothymol blue absorbance over time (FIG. 16C) and resulted in a significant percent increase in nitrogen fixation activity (FIG. 16D). All concentrations similarly increased N-fixation by 1.3× (Table 14). Iron EDTA also significantly increased bromothymol blue absorbance over time (FIG. 16C) and resulted in a significant percent increase in nitrogen fixation activity of X. autotrophicus (FIG. 16D) at concentrations greater than 5× (0.895 mM).

### Example 17: Efficacy of Molasses as a Primer for X. Autotrophicus Nitrogen Fixation

The purpose of this trial was to evaluate molasses, a source of sugars, as a primer for freeze-dried X. autotrophicus N-fixation activity.

### Experimental Design

In this experiment, primers were tested during rehydration of freeze-dried X. autotrophicus. Solutions of molasses detailed in Table 15 were prepared and 5 ml was added to 75 mg of freeze-dried X. autotrophicus. Samples were incubated at room temperature for 2 hours with intermittent mixing every 30 minutes. After 2 hours, samples were pelleted and washed of media before performing the colorimetric assay as described above.

TABLE 15
Example 17 Treatment Groups

| | | Primer | |
|---|---|---|---|
| | | Concentration | Primer |
| Treatment # | Treatment Description | (g/L) | effect |
| 1 | Freeze-dried X. | — | — |
| | autotrophicus (Control) | | |
| 2 | +0.5 g/L Molasses | 0.5 | Null |
| 3 | +1.0 g/L Molasses | 1.0 | 1.3x |
| 4 | +2.0 g/L Molasses | 2.0 | Null |
| 5 | +4.0 g/L Molasses | 4.0 | 1.5x |
| 6 | +8.0 g/L Molasses | 8.0 | Null |

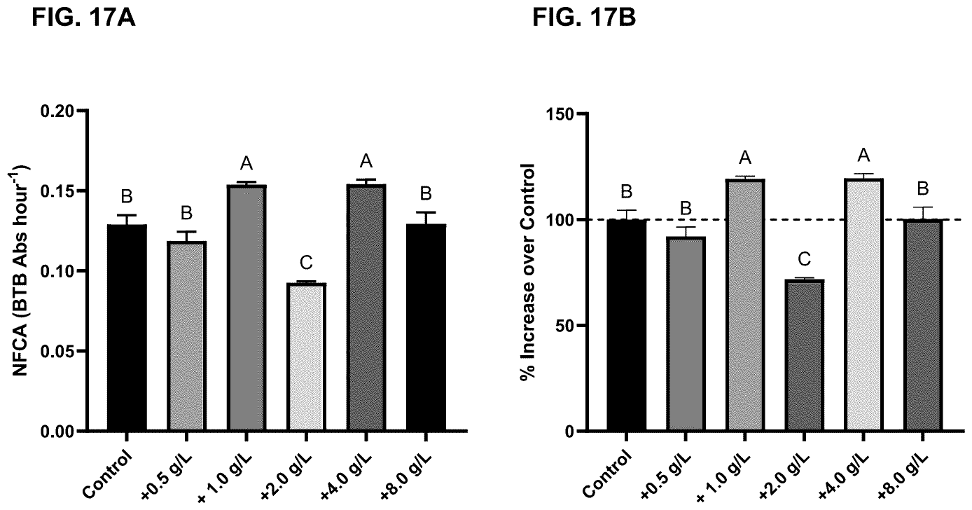

This experiment investigated the use of molasses as a primer for N-fixation activity of freeze-dried X. autotrophicus. When molasses was added at 1.0 g/L and 4.0 g/L during rehydration of freeze-dried X. autotrophicus, N-fixation rates were significantly increased by 1.3× and 1.5×, respectively (FIG. 17A). Furthermore, molasses concentrations of 1.0 g/L and 4.0 g/L each resulted in an increase in nitrogen fixation activity (FIG. 17B). No other concentrations of molasses significantly increased N-fixation activity of freeze-dried X. autotrophicus.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A composition comprising i) a biofertilizer comprising a microorganism, wherein the microorganism is Xanthobacter autotrophicus, and ii) a primer of nitrogen fixation, wherein the primer is glucose, fructose, sucrose, malic acid, pantothenic acid, thiamine, an amino acid, potassium chloride, sodium molybdate, molasses, or a combination thereof.

2. (canceled)

3. A composition comprising i) a biofertilizer comprising a microorganism, and ii) a primer of nitrogen fixation, wherein the primer is fructose, glucose, sucrose, malic acid, pantothenic acid, thiamine, an amino acid, potassium chloride, sodium molybdate, molasses, or a combination thereof.

4.-8. (canceled)

9. The composition of claim 1, wherein the primer is sucrose.

10. (canceled)

11. The composition of claim 1, wherein the primer comprises an amino acid.

12. The composition of claim 11, wherein the amino acid is glycine, glutamic acid, arginine, asparagine, tryptophan, histidine, or any combination thereof.

13.-22. (canceled)

23. The composition of claim 3, wherein the microorganism comprises Acidiphilium species, Alcaligenes species, Arthrobacter species, Azohydromonas species, Azospirillum species, Azotobacter species, Bacillus species, Beggiatoa species, Beijerinckia species, Bradyrhizobium species, Burkholderia species, Cupriavidus species, Derxia species, Herbaspirillum species, Hydrogenophaga species, Lactobacillus species, Mesorhizobium species, Methylibium species, Methylocapsa species, Methyloferula species, Methyloversatilis species, Microcyclus species, Nitrosococcus species, Nocardia species, Oligotropha species, Pannonibacter species, Paracoccus species, Pelagibaca species, Pseudomonas species, Pseudooceanicola species, Ralstonia species, Renobacter species, Rhizobium species, Rhodobacter species, Rhodomicrobium species, Rubrivivax species, Salipiger species, Sinorhizobium species, Skermanella species, Stappia species, Thauera species, Variovorax species, Xanthobacter species, or combinations thereof.

24. The composition of claim 3, wherein the microorganism comprises Acidiphilium multivorum, Alcaligenes paradoxus, Azoarcus indigens, Azohydromonas australica, Azohydromonas lata, Azorhizobium caulinodans, Azospirillium brasiliense, Azospirillum amazonsense, Azospirillum lipoferum, Azospirillum lipoferum (RSAL0111), Azospirillum thiophilum, Azotobacter chroococum (MCC 0055), Azotobacter vinelandii, Azotobacter vinelandii (RSAV006), Bacillus megaterium, Bacillus pumilus, Bacillus amyloliquefaciens, Bacillus licheniformis, Bacillus subtilis, Beggiatoa alba, Beijerinckia mobilis, Bradyrhizobium elnakii, Bradyrhizobium japonicum, Bradyrhizobium japonicum (strain USDA 122), Burkholderia vietnameiensis, Cupriavidus necator, Derxia gummosa, Gluconacetobacter diazotrophicus, Gluconacetobacter diazotrophicus (MCC 0046), Herbaspirillum autrotrophicum, Herbaspirillum frisingense (MCC 0052), Hydrogenophaga pseudoflava, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Methylibium petroleiphilum, Methylocapsa aurea, Methyloferula stellate, Methyloversatilis universalis, Microcyclus aquaticus, Microcyclus ebruneus, Nitrosococcus oceani, Nitrosomonas communis, Nitrospirillum amazonense, Nocardia autotrophica, Nocardia opaca, Oligotropha carboxidovorans, Paenibacillus durus (MCC 0046), Pannonibacter phragmitetus, Paracoccus denitrificans, Paracoccus pantrophus, Paracoccus yeei, Pelagibaca bermudensis, Pseudomonas facilis, Pseudomonas fluorescens, Pseudooceanicola atlanticus, Ralstonia eutropha, Renobacter vacuolatum, Rhizobium gallicum, Rhizobium japonicum, Rhizobium japonicum (MCC 0071), Rhizobium leguminosarum, Rhizobium leguminosarum biovar viciae, Rhodobacter capsulatus, Rhodobacter sphaeroides, Rhodomicrobium vannielii, Rubrivivax gelatinosus, Salipiger mucosus, Sinorhizobium americanum, Sinorhizobium fredii, Sinorhizobium meliloti, Skermanella stibiiresistens, Stappia aggregate, Thauera humireducens, Variovorax paradoxus, Xanthobacter autotrophicus, or combinations thereof.

25. The composition of claim 1, wherein the biofertilizer is in liquid formulation or dry formulation.

26. (canceled)

27. The composition of claim 1, wherein the primer is in liquid formulation or dry formulation.

28.-30. (canceled)

31. The composition of claim 9, wherein the composition comprises about 5% w/v of the primer.

32.-37. (canceled)

38. The composition of claim 11, wherein the amino acid is glutamic acid.

39. The composition of claim 38, wherein the composition comprises about 10% w/v to about 20% w/v of the primer.

40.-46. (canceled)

47. The composition of claim 38, wherein the composition comprises about 0.025 M to about 0.05 M of the primer or about 0.5 M to about 1.5 M of the primer.

48.-60. (canceled)

60. A kit comprising the composition of claim 1.

61.-66. (canceled)

67. The kit of claim 60, wherein the primer is sucrose.

68.-73. (canceled)

74. The kit of claim 60, wherein the primer is glutamic acid.

75.-119. (canceled)

120. A method of priming nitrogen fixation, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

121. A method of enhancing exopolysaccharides (EPS) production of a biofertilizer, comprising applying a primer to a biofertilizer comprising a microorganism, wherein the primer comprises glucose, fructose, sucrose, malic acid, pantothenic acid, thiamine, an amino acid, potassium chloride, sodium molybdate, molasses, or a combination thereof.

122. A method of enhancing microbial root colonization, comprising i) applying a primer to a biofertilizer comprising a microorganism, and ii) applying the primer and the biofertilizer to a plant, wherein the primer comprises glucose, fructose, sucrose, malic acid, pantothenic acid, thiamine, an amino acid, potassium chloride, sodium molybdate, molasses, or a combination thereof.

123. A method of increasing viability of a biofertilizer, comprising applying a primer to a biofertilizer comprising a microorganism, wherein the primer comprises glucose, fructose or a combination thereof.

124.-127. (canceled)

128. The method of claim 38, wherein the composition comprises about 0.05 g to about 10 g of primer per 1 L of the biofertilizer.
